(19) United States
(12) Patent Application Publication
(10) Pub. No.: US 2026/0284163 A1
(43) Pub. Date: Sep. 24, 2026

(54) PEPTIDE-BASED VACCINES AND METHODS FOR TREATMENT

(71) Applicant: The Johns Hopkins University, Baltimore, MD (US)

(72) Inventors: Efrosini Kokkoli, Baltimore, MD (US); Zachary Schneiderman, Baltimore, MD (US); Amanda Huff, Baltimore, MD (US); Elizabeth A. Jaffee, Baltimore, MD (US)

(21) Appl. No.: 19/358,192

(22) Filed: Oct. 14, 2025

Related U.S. Application Data
(63) Continuation of application No. PCT/US24/24544, filed on Apr. 14, 2024.
(60) Provisional application No. 63/459,458, filed on Apr. 14, 2023.

(51) Int. Cl. A61K 39/00; A61K 9/1277; A61P 37/04

(52) U.S. Cl. CPC A61K 39/0011; A61K 9/1277; A61P 37/04; A61K 2039/53; A61K 2039/852

(57) ABSTRACT

Provided are, inter alia, immunogenic and vaccine compositions including a lipid vesicle, a first nucleic acid; and a peptide, and methods of treating or preventing pancreatic cancer using the compositions.

The present application claims the benefit of U.S. provisional application No. 63/459,458 filed Apr. 14, 2023, which application is incorporated by reference herein in its entirety.

### GOVERNMENT SUPPORT

This invention was partly supported with government support under grant no. CA271470 awarded by National Cancer Institute, National Institute of Health and Department of Health and Human Services. The government has certain rights in the invention.

### SEQUENCE LISTING STATEMENT

The instant application contains a Sequence Listing which has been submitted in XML format and is hereby incorporated by reference in its entirety. Said XML copy, created on Oct. 14, 2025, is named 348358_15901 SL.xml and is 81,504 bytes in size.

### BACKGROUND

Pancreatic cancer is one of the most aggressive cancers with a short overall survival. It is often diagnosed at an advanced stage, it spreads quickly, and is usually hard to treat the cancer patient at sufficient survival rate after diagnosis. Amongst, pancreatic adenocarcinomas are less immunogenic tumors with a lower number of predicted neoantigens, low immune infiltration, and poor response rates to immune checkpoint inhibitor therapy.

It would be desirable to have new therapies to treat cancer patients, including patient suffering from pancreatic cancer.

### SUMMARY

In one aspect, we now provide new compositions and methods for delivery of neoantigen peptides, including in nanoparticle form.

Preferred immunogenic and vaccine compositions have substantial in vivo therapeutic efficacy, including reduction or elimination of tumors in treated subjects, as demonstrated in the Examples which follow.

In one aspect, an immunogenic or vaccine composition is provided that comprises a) a lipid nanoparticle comprising one or more lipid components; b) a first nucleic acid; c) a peptide; and d) one or more anchoring motifs containing one or more hydrophobic tails. The first nucleic acid forms a conjugate with the one or more anchoring motifs. In preferred embodiments, the one or more hydrophobic tails include C₅-C₃₀ alkyl group, C₅-C₃₀ alkenyl groups, or combinations thereof.

In some embodiments, the peptide is suitably conjugated to the one or more the anchor compounds. Alternatively, or in some embodiments, the peptide is suitably conjugated to the one or more lipid components. In certain preferred embodiments, the peptide is suitably conjugated to the first nucleic acid.

In certain preferred aspects, the peptide further includes a second nucleic acid that has a sequence complementary to a sequence of the first nucleic acid. Thus, the peptide and the first nucleic acid forms a hybridized complex.

In preferred aspects, the lipid vesicle is a liposome or a lipid nanoparticle. In some embodiments, the first nucleic acid is encapsulated in the lipid nanoparticle.

In preferred aspects, the first nucleic acid includes a single stranded DNA (ssDNA). In certain preferred compositions, the first nucleic acid may comprise an unmethylated cytosine (C)-phosphate (p)-guanosine (G) (CpG) oligodeoxyribonucleotide, which may be a toll-like receptor-9 (TLR-9) agonist. In certain preferred systems, the first nucleic acid comprises a sequence that has at least 80, 90 or 95 percent identify to SEQ ID NO: 5, SEQ ID NO: 6 or SEQ ID NO: 7. In a particular preferred composition, the first nucleic acid comprises SEQ ID NO: 5, SEQ ID NO: 6 or SEQ ID NO: 7.

In certain preferred systems, the first nucleic acid comprises a sequence that has at least 80, 90 or 95 percent identify to SEQ ID NO: 8. In a particular preferred composition, the first nucleic acid comprises SEQ ID NO: 8, which also can bind to the DEC-205 receptor on dendritic cells (DCs).

In certain preferred compositions, the first nucleic acid may be conjugated or attached to one or more anchor compounds, via hydrophobic tails including one or more C₅-C₃₀ alkyl and C₅-C₃₀ alkenyl group.

In certain preferred compositions, the peptide comprises a neoantigen or a tumor cell neoepitope, for example the neoepitope of a pancreatic tumor cell such as a pancreatic adenocarcinoma cell. The peptide suitably may be synthetic peptide. Preferred peptides include those that contain at least 8, 10, 12, 14, 15, 20, 25, 30, 35, 40, 45 or 50 amino acids, such from 10 to 35, 40, 45 or 50 or 15 to 25 or 30 amino acids. Particularly preferred peptides may include a sequence of a neoantigen or neoepitope present on a pancreatic tumor cell.

In certain preferred compositions the first nucleic acid is hybridized with the peptide via the second nucleic acid, or conjugated directly to the peptide.

In certain preferred compositions, the peptide may be suitably conjugated to the one or more anchor compounds, e.g., via chemical linkage to, or hydrophobic interaction with the hydrophobic tails including the one or more C₅-C₃₀ alkyl and C₅-C₃₀ alkenyl group.

In certain preferred compositions, the lipid vesicle has a size ranging from about 50 to about 200 nm. In certain compositions, suitably, a ratio of the first nucleic acid and the lipid vesicle ranges from about 1:1 to about 1000:1.

In further aspects, provided are methods of preparing the immunogenic and vaccine compositions as described herein.

In an aspect, provided is a method of treating or preventing a cancer in a subject. The method includes administering the composition as described herein. In certain embodiments, the cancer is a pancreatic cancer or pancreatic adenocarcinoma.

In an aspect, provided is a kit including an immunogenic or vaccine composition as described herein.

Other aspects of the invention are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C show that CpG decorated lipid nanoparticles (LNP) have improved uptake by murine bone marrow derived dendritic cells (BMDCs). FIG. 1A: Schematic for LNP-CpG decorated nanoparticle uptake assay in murine antigen presenting cells, BMDCs. On day 0, bone marrow from C57BL/6 mice was isolated and cultured with 20 ng/mL mGMCSF for 5 days. BMDCs were then collected and cultured for 2 hours with 2.5 μM, 1 μM, or 0.1 μM non-decorated LNP or CpG decorated LNP (LNP-CpG) encapsulating sulfo-Cy5 dye. Nanoparticles were then removed and cells were washed with PBS. LNP-pulsed cells were collected immediately after wash or 24 hours later and analyzed by flow cytometry for sulfo-cy5 dye uptake in dendritic cells (CD11c, MHCII) with activation markers (CD86, CD40). FIG. 1B: Dendritic cell population (CD11c+MHCII+) gated on for LNP uptake analysis. FIG. 1C: Detection of sulfo-Cy5 labeled LNPs in the dendritic cell population at 2 hours (left) and 24 hours (right) after incubation with the indicated concentrations of LNPs. CpG decorated LNPs demonstrate increased uptake in BMDCs compared to non-decorated particles.

FIGS. 2A-2D show that BMDCs preferentially uptake CpG decorated lipid nanoparticles (LNP-CpG) as measured by higher percent of sulfo-Cy5 labeled cells and median fluorescent intensity (MFI) increase in sulfo-cy5 stained cells. Flow cytometry analysis of sulfo-cy5 uptake in CD11c+MHCII+BMDCs after 2.5 μM, 1 μM, 0.1 μM LNP or LNP-CpG culture as indicated in FIGS. 1A-ID. FIG. 2A: Percent sulfo-cy5+BMDCs 2 hours, FIG. 2B: 24 hours after LNP or LNP-CpG incubation. Untreated and LPS treated BMDCs are used as negative and positive controls, respectively. FIG. 2C: MFI of sulfo-cy5 dye in BMDCs 2 hours or FIG. 2D: 24 hours after LNP or LNP-CpG incubation. MFI represents the fluorescence value at which half of a measured cell population is above and the other half below. One way ANOVA followed by Sidak's multiple comparisons, ns=p>0.05, *p<0.05, **p<0.005, ***p<0.0005, ****p<0.00001.

FIGS. 3A-3B show that CpG decorated nanoparticles (LNP-CpG) significantly enhance BMDC activation and costimulatory molecule expression in vitro. Flow cytometry analysis of activation marker expression in CD11c+MHCII+ BMDCs after 2.5 μM, 1 μM, 0.1 μM LNP or LNP-CpG culture as indicated in FIGS. 1A-1D. FIG. 3A: Median fluorescent intensity (MFI) of activation marker CD86 expression 24 hours after LNP or LNP-CpG culture. Untreated cells serve as negative control while LPS treated BMDCs serve as positive control for BMDC activation. FIG. 3B: MFI of activation marker CD40 expression 24 hours after LNP or LNP-CpG culture. One way ANOVA followed by Sidak's multiple comparisons, ns=p>0.05, *p<0.05, **p<0.005, ***p<0.0005, ****p<0.00001

FIGS. 4A-4C show that dendritic cells and macrophages preferentially took up CpG-nanoparticles (LNP-CpG) from whole splenocyte cultures in vitro. FIG. 4A: Schematic for LNP-CpG decorated nanoparticle uptake assay in murine splenocytes. On day 0, mouse spleens were isolated and processed to single cell suspension. Whole splenocytes were cultured for 2 hours with 2.5 μM, 1 μM, or 0.1 μM non-decorated LNP or CpG decorated LNP (LNP-CpG) encapsulating sulfo-Cy5. Nanoparticles were then removed and cells were washed with PBS. LNP-pulsed cells were collected immediately after wash or 24 hours later and analyzed by flow cytometry for sulfo-cy5 dye uptake. FIG. 4B: Splenocyte cell type populations analyzed for LNP uptake including T cells (CD3+), B cells (CD19+), dendritic cells (CD11c+MHCII+), and macrophages (CD11b+F4/80+). FIG. 4C: Sulfo-Cy5 median fluorescent intensity (MFI) in each splenocyte cell type after 2 hours (left) or 24 hours (right) post culture. CpG decorated particles (LNP-CpG) had significantly improved uptake in all cell types compared to non-CpG particles (LNP). Two way ANOVA followed by Tukey's multiple comparisons, ns=p>0.05, *p<0.05, **p<0.005, ***p<0.0005, ****p<0.00001

FIGS. 5A-5C show that LNP-CpG-44/237 peptide vaccine induces robust epitope-specific T cell responses in vivo. FIG. 5A: In vivo immunogenicity testing of CpG decorated LNP (LNP-CpG) carrying murine pancreatic cancer line Panc02 neoantigens 44 (peptide having SEQ ID NO: 1) and 237 (peptide having SEQ ID NO: 2). Mice were vaccinated on day 0 and day 7 with PBS control, LNP-CpG control, or LNP-CpG-44/237 at 20 μg (10 μg/peptide), 10 μg (5 μg/peptide) and 1 μg (0.5 μg/peptide). On day 14, spleens were collected from vaccinated mice and used for enzyme-linked immunospot (ELISPOT). FIG. 5B: Amino acid sequences of peptides 44 and 237. The mutated residue is shown in red. Figure disclose SEQ ID NOS 1 and 2, respectively, in order of appearance. FIG. 5C: IFNγ production by CD8+ T cells from vaccinated mice after overnight incubation with T2-H2-Kb and T2-H2-Db antigen presenting cells pulsed with control peptide 44 and 237 measured by ELISPOT. Two way ANOVA followed by Tukey's multiple comparisons, ns=p>0.05, *p<0.05, **p<0.005, ***p<0.0005, ****p<0.00001

FIGS. 6A-6C show that splenic dendritic cells express significantly more costimulatory molecules 48 hours after LNP-CpG vaccination. FIG. 6A: In vivo immunogenicity testing of CpG decorated LNP (LNP-CpG) carrying murine pancreatic cancer line Panc02 neoantigens 44 and 237. Mice were implanted with 3×10⁶ panc02 cells, subcutaneously. 21 days later, mice were vaccinated with PBS control, LNP-CpG, or LNP-CpG-44/237 (5 μg/peptide), subcutaneously. Splenocytes and tumors were collected 48 hours or 7 days post vaccination and analyzed by flow cytometry for myeloid and lymphoid cell activation. FIG. 6B: Gating scheme for dendritic cell (CD11c+MHCII+) compartment in spleens 48 hours post vaccination. FIG. 6C: Percent dendritic cell population (left) and costimulatory molecule CD86 expression across treatment groups. Fluorescence minus one (FMO) control with no CD86 antibody used as technical negative control. One-way ANOVA followed by Sidaks's multiple comparisons, ns=p>0.05, *p<0.05, **p<0.005, ***p<0.0005, ****p<0.00001

FIG. 7A-7D show that LNP-CpG-44/237 peptide vaccination results in significantly more activated CD8+ T cell infiltration in the tumor compared to LNP-CpG control. FIG. 7A: Flow cytometry of LNP-CpG-44/237 vaccinated tumor bearing mice as in FIG. 6A. Gating scheme for identification of NK cells (NK1.1+), CD19 cells (CD19+), T cells (NK1.1−CD19−CD3+) and CD4+ or CD8+ T cells. FIG. 7B: Cell counts of each cell type per mg of tumor identified by flow cytometry 7 days post vaccination. FIG. 7C: Number of activated CD8+ T cells (CD3+CD8+CD44+) per mg of tumor in all treatment groups. FIG. 7D: Number of inhibitory receptor expressing CD8+ T cells per mg of tumor in all treatment groups. One-way ANOVA followed by Sidaks's multiple comparisons, ns=p>0.05, *p<0.05, **p<0.005, ***p<0.0005, ****p<0.00001

FIGS. 8A-8B show that LNP-CpG-peptide vaccine significantly improves control of tumor growth. Therapeutic efficacy analysis of LNP-CpG-44/237 vaccine in the murine panc02 model. FIG. 8A: Mice were implanted with 3×10⁶ panc02 cells, subcutaneously. On day 3 and day 7, mice receive PBS, LNP-CpG control, or LNP-CpG-44/237 vaccine at a dose of 10 μg, 5 μg, or 1 μg per peptide. FIG. 8B: Tumors were monitored for growth.

FIGS. 9A-9D show that LNP-CpG-peptide vaccine clears tumors in 5/10 mice treated. Therapeutic efficacy analysis of LNP-CpG-44 vaccine in the murine panc02 model compared to peptide vaccine. FIG. 9A: Mice were implanted with 3×10⁶ panc02 cells, subcutaneously. On day 3 and day 7, mice receive PBS, 44 peptide based vaccine (Addavax+ADUS100 agonist), LNP-CpG control, or LNP-CpG-44 (10 μg/peptide). FIG. 9B: Tumors were monitored for growth. FIG. 9C: Percentage of mice tumor free at day 50 post treatment. FIG. 9D: Survival curves of mice that received different treatments. Statistical significance, ns=p>0.05, *p<0.05.

FIG. 10 shows successful post-insertion of CpG-amphiphiles into liposomes at different ratios of CpG-amphiphiles to individual liposomes. Strong bands at the top of the wells show that the large majority of the CpG-amphiphiles were successfully post-inserted at all ratios.

FIG. 11 shows exemplary compounds containing hydrophobic (dialkyl) tails used to form a conjugate of the first nucleic acid sequence or peptide (n=10-22).

FIG. 12A-12F show exemplary anchoring motifs and conjugates for various vaccine designs.

FIG. 13 IFNγ production by CD8+ T cells from vaccinated mice after overnight incubation with T2-H2-Kb and T2-H2-Db antigen presenting cells pulsed with vaccine designs shown in FIG. 12 measured by ELISPOT. Two way ANOVA followed by Tukey's multiple comparisons, ****p<0.00001.

### DETAILED DESCRIPTION

We provide new immunogenic compositions delivery systems for treatment and prevention of a cancer such as pancreatic cancer. In one aspect, targeted delivery of immunogenic compositions is provided that comprise inserting a neoantigen peptide into a lipid vehicle (e.g., liposome or lipid nanoparticle) and forming a nanoparticle including vaccination components (e.g., adjuvant, neoepitope and the like).

As discussed, the present immunogenic and vaccine compositions can provide substantial in vivo therapeutic efficacy, including reduction or elimination of tumors in treated subjects, as demonstrated in the Examples which follow.

### Definitions

The abbreviations used herein have their conventional meaning within the chemical and biological arts. The chemical structures and formulae set forth herein are constructed according to the standard rules of chemical valency known in the chemical arts.

In this specification and the appended claims, the singular forms “a,” “an” and “the” include plural referents unless the context clearly dictates otherwise. The terms “a” (or “an”), as well as the terms “one or more,” and “at least one” can be used interchangeably herein.

Wherever aspects are described herein with the language “comprising,” otherwise analogous aspects described in terms of “consisting of” and/or “consisting essentially of” are also provided.

Unless otherwise indicated, all numbers, values, and/or expressions referring to quantities of ingredients, reaction conditions, polymer compositions, and formulations used herein are to be understood as modified in all instances by the term “about” as such numbers are inherently approximations that are reflective of, among other things, the various uncertainties of measurement encountered in obtaining such values. Further, unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the disclosure when a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. The term “treating” and conjugations thereof, may include prevention of an injury, pathology, condition, or disease. In embodiments, treating is preventing. In embodiments, the term “treating” does not include preventing.

The term “prevent” refers to a decrease in the occurrence of disease (e.g., cancer) symptoms in a patient. As indicated above, the prevention may be complete (e.g., no detectable symptoms) or partial, such that fewer symptoms are observed than would likely occur absent treatment.

As used herein, the term “cancer” refers to all types of cancer, neoplasm, or malignant tumors found in mammals, including leukemia, lymphomas, carcinomas and sarcomas. Exemplary cancers include acute myeloid leukemia (“AML”), chronic myelogenous leukemia (“CML”), and cancer of the brain, breast, pancreas, colon, liver, kidney, lung, non-small cell lung, melanoma, ovary, sarcoma, and prostate. Additional examples include, cervix cancers, stomach cancers, head & neck cancers, uterus cancers, mesothelioma, metastatic bone cancer, Medulloblastoma, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, uterine cancer, urinary bladder cancer, bladder cancer, esophagus cancer, gastric cancer, head and neck cancer, cholangiocarcinoma, mesothelioma, premalignant skin lesions, testicular cancer, lymphomas, thyroid cancer, neuroblastoma, esophageal cancer, genitourinary tract cancer, malignant hypercalcemia, endometrial cancer, adrenal cortical cancer, fallopian tube cancer, neoplasms of the endocrine and exocrine pancreas cancer, prostate cancer, breast cancer including triple negative breast cancer, and cutaneous T-cell lymphoma.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “associated,” “conjugated” and “attached,” when used with respect to two or more components, refers that the components are physically or chemically associated or connected, either directly (e.g., covalent bonding between two components) or via one or more additional a linking moiety or agent to form a complex that may be sufficiently stable as being conjugated or attached under the conditions in which the structure is used, e.g., physiological conditions. In certain embodiments, ionic or hydrogen bonding, or a hybridization interaction (e.g., between complementary nucleic acids) can form physically or physiologically stable conjugation or attachment, association.

As used herein, the term “contacting” or “hybridizing” means an action of establishing a physical connection between two or more components. In certain embodiments, biological molecules (e.g., nucleic acids or nucleotides) can be contacted or hybridized to allow make a physical or chemical interaction between the components or compounds. In certain embodiments, a first nucleotide can be hybridized with a second nucleotide having a complementary sequence to that of the first nucleotide.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

An “effective amount” is an amount sufficient for a composition (e.g., vaccine composition) to accomplish a stated purpose relative to the absence of the composition (e.g., achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.”

The term “therapeutically effective amount,” as used herein, refers to that amount of the therapeutic agent sufficient to treat or present a disease (e.g., cancer), as described above. For example, a therapeutically effective amount can have at least a 1.2-fold, 1.5-fold, 2-fold, 5-fold, or more effect over a control. As is well known in the art, therapeutically effective amounts for use in humans can also be determined from animal models. For example, a dose for humans can be formulated to achieve a concentration that has been found to be effective in animals. The dosage in humans can be adjusted by monitoring vaccine effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the vaccine being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the vaccine. Thereafter, the dosage is increased by small increments until the optimum effect under circumstances is reached. Dosage amounts and intervals can be adjusted individually to provide levels of the administered vaccine effective for the particular clinical indication being treated. This will provide a therapeutic regimen that is commensurate with the severity of the individual's disease state.

As used herein, a “formulation” includes at least a polynucleotide and one or more of a carrier, an excipient, and a delivery agent.

A “fragment,” as used herein, refers to a portion. In certain embodiments, a peptide refers to a fragment of a protein that may be obtained by digesting full-length protein isolated from cultured cells or by synthesizing such fragment according to the sequence identified or provided in the data base. In certain embodiments, a fragment is a portion having specific function or interest and excised from the whole sequence of the protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe, cell, or tissue thereof).

As generally defined in the art and used herein, the term “lipid” refers to a small molecule or compound that has one or more hydrophobic or amphiphilic moieties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, polyketides, and prenol lipids. In certain embodiments, the lipid may be suitably functionalized, for example, by adding a functional group or chemical group, a small molecule, a polymer, a peptide, or a ligand to the lipid. The amphiphilic properties of some lipids lead them to form liposomes, vesicles, or membranes in aqueous media.

The term “nucleic acid,” in its broadest sense, includes any compound and/or substance that comprises a polymer of nucleotides. Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil. The nucleic acids are often referred to as polynucleotides. Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a (3-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

The term “polynucleotide” as used herein refers to a biologically active polymer make of nucleotides of any length, including ribonucleotides, deoxyribonucleotides, modified analogs thereof, or mixtures thereof. The term “oligonucleotide” refers to a short form of polynucleotide, e.g., containing two to thirty nucleotides. The polynucleotide or oligonucleotide may be a synthetic material, e.g., a synthetic RNA or a synthetic DNA, includes natural nucleobases, i.e., A (adenosine), G (guanosine), C (cytidine), and T (thymidine) in the case of a synthetic DNA, or A, C, G, and U (uridine) in the case of a synthetic RNA. In certain embodiments, these terms include triple-, double- and single-stranded deoxyribonucleic acid (“DNA”), as well as triple-, double- and single-stranded ribonucleic acid (“RNA”). In certain embodiments, these terms include also include modified, for example by alkylated, or dealkylate, and unmodified forms of the polynucleotide or oligonucleotide. In certain embodiments of the disclosure, the polynucleotide or oligonucleotide includes a single stranded DNA (ss-DNA).

The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., ss-DNA.

The “first” component and “second” component may be the same or different type. In certain embodiments, the first nucleic acid and the second nucleic acid may be the same or different in sequences or size thereof. In certain embodiments, the first nucleic acid and the second nucleic acid may be different in sequences or in size.

The term “peptide”, as used herein, refers to proteins or fragment thereof, or synthetic compounds made of amino acids in any size, structure, or function. The peptide includes an encoded polynucleotide product, naturally occurring polypeptides, synthetic polypeptides, homologs, orthologs, paralogs, fragments and other equivalents, variants, and modified analogs of the foregoing. In certain embodiments, the “peptide” may be less than or equal to 50 amino acids long, e.g., about 5, 10, 15, 20, 25, 30, 35, 40, 45, or 50 amino acids long.

The term “anchoring motif”, as used herein, refers to a motif or a part of a complex that includes substantially hydrophobic region (e.g., hydrophobic tail) so it can be readily embedded into the lipid layer of a lipid vesicle (e.g., lipid nanoparticles) without addition of any surfactant or physical treatment (e.g., sonication, mixing, or perturbation) via hydrophobic interaction. In certain embodiments, the anchoring motif contains one or more long hydrocarbon chains, e.g., C₅-C₃₀ alkyl group, C₅-C₃₀ alkenyl groups, which impart hydrophobicity for anchoring into the lipid layer.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the vaccines of the disclosure. One of skill in the art will recognize that other pharmaceutical excipients are useful in the disclosure.

As used herein “stable” refers to a compound or composition (e.g., vaccine) that is sufficiently robust to survive solely or in a reaction mixture, for example, in a storage condition of pharmaceutical formulation or in physiological condition after being administered to a subjected.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

As used herein, the term “adjuvant” refers to a substance that can promote, increase or modulate immune response in a subject in exposure to an immunogenic composition or vaccine. In certain embodiments, the adjuvant may include a motif or ligand recognizing or binding to toll-like receptors (TLRs) to elevate innate immune responses in exposure to a vaccine. In certain embodiments, the vaccine may include a toll-like receptor-9 (TLR-9) agonist. In certain embodiments, the vaccine may include CpG-amphiphile as adjuvant. In certain embodiments, the vaccine may include one or more adjuvants.

As used herein, the term “neoantigen” refers to a type of antigen present on a tumor or cancer cell but absent from a normal human cell or its genome. The neoantigen is typically encoded by a mutant gene in the tumor and cancer cell. As used herein, the term “neoepitope” refers to a part or fragment serving as antigenic determinant of a neoantigen, for example, which can be recognized by immune cells (e.g., T cells). In some embodiments, the neoepitope may include a major mutant different from a normal protein or marker.

As used herein, the term “synthetic” means produced, prepared, and/or manufactured by the hand of man. For example, synthetic nucleic acid or peptide can be obtained by chemical or enzymatic (e.g., in vivo or in vitro) means.

As used herein, “targeted” refers to any one or more cells of interest. In certain embodiments, the targeted cells can be found in vitro, in vivo, in situ or in the tissue or organ of an organism, subject or a patient.

“Inducing an immune response” may mean that there was no immune response before induction, but it may also mean that there was a certain level of immune response before induction and after induction said immune response is enhanced. Thus, “inducing an immune response” also includes “enhancing an immune response”. Preferably, after inducing an immune response in a subject, the subject is protected from developing a disease such as a cancer disease or the disease condition is ameliorated by inducing an immune response. For example, an immune response against a tumor-expressed antigen may be induced in a patient having a cancer disease or in a subject being at risk of developing a cancer disease. Inducing an immune response in this case may mean that the disease condition of the subject is ameliorated, that the subject does not develop metastases, or that the subject being at risk of developing a cancer disease does not develop a cancer disease.

### Immunogenic and Vaccine Compositions

As discussed, provided herein are immunogenic and vaccine composition that include a) a lipid vesicle comprising one or more lipid components each of which is optionally functionalized; b) a first nucleic acid; c) a peptide; and d) one or more anchoring motifs containing one or more hydrophobic tails. The first nucleic acid forms a conjugate with the one or more anchoring motifs. The one or more hydrophobic tails includes C₅-C₃₀ alkyl group, C₅-C₃₀alkenyl groups, or combinations thereof.

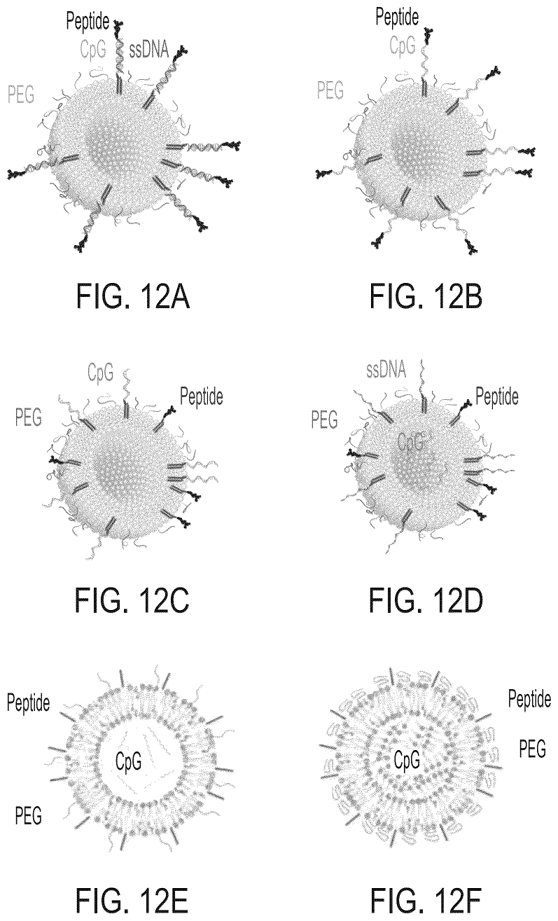

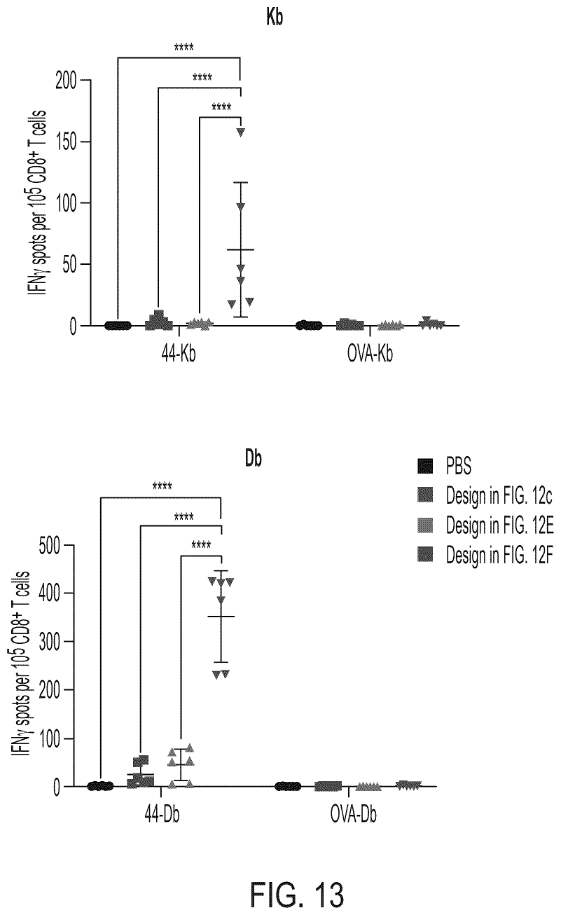

In certain embodiments, the peptide may be suitably conjugated (directly or indirectly) to the one or more the anchor compounds (FIGS. 12C-F). In certain embodiments, the peptide may be suitably conjugated (directly or indirectly) to the one or more lipid components.

In certain preferred aspects, the peptide may be suitably conjugated to the first nucleic acid (FIG. 12B). In certain aspects, the peptide further includes a second nucleic acid that has a sequence complementary to a sequence of the first nucleic acid (FIG. 12A). Preferably, the peptide and the first nucleic acid can form a hybridized complex.

In preferred aspects, lipid vesicle is a liposome or a lipid nanoparticle. In some embodiments, the first nucleic acid may be encapsulated in the lipid nanoparticle (FIGS. 12D-F).

In some embodiments, the first nucleic acid may be directly or indirectly conjugated to one or more the anchor compounds. In certain embodiments, the peptide may be conjugated to the one or more anchoring motifs, e.g., via chemical bonding or hydrophobic interaction.

In some embodiments, the peptide may be directly or indirectly conjugated to the first nucleic acid. In certain preferred embodiments, the peptide may include a second nucleic acid that has a sequence complementary to a sequence of the first nucleic acid. The peptide may form a hybrid complex with the first nucleic acid.

In some embodiments, the one or more lipid components include phospholipid. The lipid component may be suitably functionalized with a functional group, a small molecule, polymer, a peptide, a ligand, or combinations thereof.

In some embodiments, the lipid vesicle is a liposome or a lipid nanoparticle. For example, the lipid vesicle (e.g., lipid nanoparticle) has a size ranging from about 1 to about 900 nm, from about 10 to about 800 nm, from about 10 to about 700 nm, from about 10 to about 600 nm, from about 10 to about 500 nm, from about 10 to about 400 nm, from about 10 to about 300 nm, or from about 10 to about 200 nm. In certain embodiments, the lipid vesicle has a size ranging from about 50 to about 100 nm, or from about 100 to 150 nm.

In some embodiments, the lipid vesicle forms a lipid nanoparticle together with nucleic acid components (e.g., a first nucleic acid and peptide). In the disclosure, the term “lipid nanoparticle” refers to a lipid vesicle that incorporates the first nucleic acid and the conjugate described herein.

In some embodiments, the lipid vesicle has a zeta potential (mV) ranging from about −50 to 50 mV. In some embodiments, the lipid vesicle prior to incorporating other components (nucleic acid components) has the zeta potential (mV) ranging from about −50 to about +50 mV. In some embodiments, the lipid vesicle after incorporating other components (nucleic acid components) has the zeta potential (mV) ranging from about −50 to about 50 mV. The nucleic acids and peptides on the surface of the lipid vesicle alter the surface charge of the nanoparticle, e.g., by exposing additional anionic charges from the backbone of the nucleic acids or the peptides, or cationic charges from the peptides. In some embodiments, the zeta potential of the nanoparticle may be controlled by adjusting the amounts of the nucleic acids or peptides or complex incorporated in accordance of physical and chemical stability of the vaccine compositions.

The lipid nanoparticles can be generated using components, compositions, and methods as are generally known in the art, for example, WO2013123523, WO2012170930, WO2011127255, WO2008103276; or U.S. Pub. No. US20130171646, each of which is herein incorporated by reference in its entirety.

In some embodiments, the lipid vesicle includes a phospholipid, a structural lipid, and a molecule capable of reducing particle aggregation, for example a PEG, PEO, PEO-modified lipid or PEG-modified lipid. In some embodiments, the lipid vesicle includes an ionizable cationic lipid (e.g., a cationic lipid containing amines or amine alcohol). Exemplary cationic lipid may include, but not be limited to, DLin-DMA, DLin-D-DMA, DLin-MC3-DMA, DLin-KC2-DMA, DODMA, DOTAP, SM-102, L319, MC3, ALC-0315 and amino alcohol lipids. In some embodiments, the lipid vesicle includes a non-cationic lipid. In some embodiments, the lipid vesicle includes PEG-modified lipid.

In some embodiments, the lipid vesicle includes a phospholipid in a molar ratio of 80%, 81%, 82%, 83%, 84%, 85%, 86%, 87%, 89%, 90%, 91%, 93%, 94%, 95%, 96%, 97%, 98%, 99%, 99.5% of the lipid vesicle.

In some embodiments, the lipid vesicle includes a PEG-modified lipid in a molar ratio of 0.5%, 1%, 2%, 3%, 4%, 5%, 6%, 7%, 8%, 9%, or 10% of the lipid vesicle. Exemplary modified PEG lipid includes DMG-PEG, DSG-PEG, DOPE-PEG, DOPE-PEG-amine, DOPE-PEG-COOH, DOPE-PEG-Mal, M-PEG-DMPE, M-PEG-DOPE, DSPE-PEG, DSPE-PEG-Amine, DSPE-PEG-Acid, DSPE-PEG-COOH, DSPE-PEG-CH2COOH, DSPE-PEG-NHS, DSPE-PEG-Azide, DSPE-PEG-DBCO, DSPE-PEG-Aldehyde, DSPE-PEG-Biotin, DSPE-PEG-Folate, DSPE-PEG-OH, DSPE-PEG-SH, DSPE-PEG-SPDP, DSPE-PEG-IA, DSPE-PEG-Vinylsulfone, DSPE-PEG-Cy3, DSPE-PEG-Cy5, DSPE-PEG-FITC, DSPE-PEG-Rhodamine, M-PEG-DSPE, DPPE-PEG, DPPE-PEG-Amine, DPPE-PEG-Acid, DPPE-PEG-COOH, DPPE-PEG-CH2COOH, DPPE-PEG-NHS, DPPE-PEG-Azide, DPPE-PEG-DBCO, DPPE-PEG-Aldehyde, DPPE-PEG-Biotin, DPPE-PEG-Folate, DPPE-PEG-OH, DPPE-PEG-SH, DPPE-PEG-SPDP, DPPE-PEG-IA, DPPE-PEG-Vinylsulfone, DPPE-PEG-Cy3, DPPE-PEG-Cy5, DPPE-PEG-FITC, DPPE-PEG-Rhodamine, M-PEG-DPPE which may be supplied by commercial sources.

In some embodiments, the lipid vesicle includes a cationic lipid containing amine alcohol in a molar ratio of 0.5%, 1%, 2%, 3%, 4%, 5%, 6%, 7%, 8%, 9%, or 10% of the lipid vesicle. The cationic lipid containing amino alcohol may include, but not be limited to, lipids described in and/or made by the methods described in U.S. Publication No. US20130150625, herein incorporated by reference in its entirety. Exemplary cationic lipid may include, but not be limited to, (20Z,23Z)-N,N-dimethylnonacosa-20,23-dien-10-amine, (17Z,20Z)-N,N-dimemylhexacosa-17,20-dien-9-amine, (1 Z,19Z)-N,N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21 Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimethyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11 Z,14Z)-1-nonylicosa-11,14-dien-1-yl]pyrrolidine, (20Z)-N,N-dimethylheptacos-20-en-10-amine, (15Z)-N,N-dimethylheptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1 S,2R)-2-octylcyclopropyl]heptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11 Z,14Z)-icosa-11,14-dien-1-yloxy]-N,N-dimethyl-3-(pentyloxy)propan-2-amine, (2S)-1-(hexyloxy)-3-[(11 Z,14Z)-icosa-11,14-dien-1-yloxy]-N,N-dimethylpropan-2-amine, 1-[(11 Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9ZJ 2Z)-octadeca-9J 2-dien-1-yloxy]propan-2-amine, (2R)-1-[(3J-dimethyloctyl)oxy]-N,N-dimethyl-3 (9ZJ 2Z)-octadeca-9J 2-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some embodiments, the one or more functionalized lipid components include one or more selected from the group consisting of polyethylene glycol (PEG); dipalmitoylphosphatidylcholine (DPPC); distearoylphosphatidylcholine (DSPC); DPPE, DSPE; 1,2-distearoyl-sn-glycero-3-phosphoethanolamine-N-[amino(polyethylene glycol)](DSPE-PEG amine); 1,2-dipalmitoyl-sn-glycero-3-phosphoethanolamine-N-[4-(p-maleimidomethyl)cyclohexane-carboxamide]; 1,2-dioleoyl-sn-glycero-3-phosphoethanolamine-N-[4-(p-maleimidomethyl)cyclohexane-carboxamide]; 1,2-distearoyl-sn-glycero-3-phosphocholine (N-aminoethyl); 1,2-dioleoyl-sn-glycero-3-phosphocholine (N-aminoethyl); 1,2-dioleoyl-sn-glycero-3-phosphoethanolamine-N-[4-(p-maleimidophenyl)butyramide]; 1,2-dipalmitoyl-sn-glycero-3-phosphoethanolamine-N-[4-(p-maleimidophenyl)butyramide]; 1,2-dioleoyl-sn-glycero-3-phosphoethanolamine-N-(hexanoylamine); 1,2-dipalmitoyl-sn-glycero-3-Phosphoethanolamine-N-(hexanoylamine); 1,2-dipalmitoyl-sn-glycero-3-phosphothioethanol; 1,2-dioleoyl-sn-glycero-3-phosphoethanolamine-N-[3-(2-pyridyldithio)propionate]; 1,2-dipalmitoyl-sn-glycero-3-phosphoethanolamine-N-[3-(2-pyridyldithio)propionate]; 1,2-dioleoyl-sn-glycero-3-phosphoethanolamine-N-(succinyl); 1,2-dipalmitoyl-sn-glycero-3-phosphoethanolamine-N-(succinyl); 1,2-dioleoyl-sn-glycero-3-phosphoethanolamine-N-(glutaryl); 1,2-dipalmitoyl-sn-glycero-3-phosphoethanolamine-N-(glutaryl); 1,2-dioleoyl-sn-glycero-3-phosphoethanolamine-N-(dodecanoyl); 1,2-dioleoyl-sn-glycero-3-phosphoethanolamine-N-(cap biotinyl); 1,2-dipalmitoyl-sn-glycero-3-phosphoethanolamine-N-(cap biotinyl); 1,2-dipalmitoyl-sn-glycero-3-phosphoethanolamine-N-(cyanur); 1,2-dipalmitoyl-sn-glycero-3-phosphoethanolamine-N-(6-((folate)amino)hexanoyl); 1,2-dipalmitoyl-sn-glycero-3-phospho((ethyl-1′,2′,3′-triazole)triethyleneglycolmannose); 1,2-distearoyl-sn-glycero-3-phosphoethanolamine-N-square; 1,2-dioleoyl-sn-glycero-3-phospho(ethylene glycol); 1,2-dipalmitoyl-sn-glycero-3-phospho(ethylene glycol); and 1,2-distearoyl-sn-glycero-3-phosphoethanolamine-N-[(polyethylene glycol)-2000]-N-[4-(triphenylphosphonium) butanamide].

In some embodiments, the lipid nanoparticles include about 90 to 99.5 mol % DPPC or DSPC and about 0.5 to 10 mol % of PEGylated lipids that include DSPE-PEG750, or DSPE-PEG1000, or DSPE-PEG2000, or DSPE-PEG3000 or DPPE-PEG750, or DPPE-PEG1000, or DPPE-PEG2000, or DPPE-PEG3000. In some embodiments, the lipid nanoparticles include about 95 to 99 mol % DPPC or DSPC and about 5 to 10 mol % PEGylated lipids. In some embodiments, the lipid nanoparticles include about 98 to 99 mol % DPPC or DSPC and about 1 to 2 mol % PEGylated lipids.

In some embodiments, the vaccine composition includes one or more nucleic acids. Particularly, the immunogenic or vaccine composition disclosed herein includes a first nucleic acid (e.g., single stranded DNA) formulated in the same lipid nanoparticle.

In some embodiments, the CpG oligodeoxyribonucleotide is a toll-like receptor-9 (TLR-9) agonist. In some embodiments, the first nucleic acid comprises an unmethylated cytosine (C)-phosphate (p)-guanosine (G) (CpG) oligodeoxyribonucleotide. The CpG may be suitably conjugated to a hydrophobic tail (“CpG-amphiphiles”) or is encapsulated in the nanoparticle. For example, the first nucleic acid may be conjugated or attached to the one or more C₅-C₃₀ alkyl or C₅-C₃₀ alkenyl group (FIG. 12).

Preferably, the CpG oligodeoxyribonucleotide binds to the DEC-205 receptor on dendritic cells (DCs). For example, the first nucleic acid has a sequence of SEQ ID NO: 5, SEQ ID NO: 6 or SEQ ID NO: 7. Exemplary CpG-amphiphiles may include, but not be limited to, compounds described in and/or made by the methods described in U.S. Publication No. WO2019169328, U.S. Pat. No. 9,314,521, WO2019169328, and Kinkead et al., JCI Insight. 2018, each of which is herein incorporated by reference in its entirety. Preferably, the first nucleic acid having the sequence of SEQ ID NO: 5, SEQ ID NO: 6 or SEQ ID NO: 7 can bind to the DEC-205 receptor on dendritic cells (DCs). In some embodiments, the first nucleic acid may have poly-T sequences (e.g., SEQ ID NO: 8), which can also bind to the DEC-205 receptor on dendritic cells (DCs).

In some embodiments, the peptide comprises a neoantigen or a neoepitope present on a tumor cell, or a fragment thereof. In particular, the tumor cell is a pancreatic tumor cell, or a pancreatic adenocarcinoma cell. In some embodiments, the peptide is a synthetic long peptide including more than 10 amino acids, more than 15 amino acids, more than 10 amino acids, more than 25 amino acids, more than 30 amino acids, or more than 35 amino acids. In some embodiments, the peptide is a synthetic long peptide including 10 to 40 amino acids, or 15 to 25 amino acids. In certain embodiments, the peptide has an amino acid sequence of SEQ ID NO: 1, SEQ ID NO: 2, or combination thereof. In some embodiment, the peptide used and incorporated in the nanoparticle may be a mixture of the peptide of SEQ ID NO: 1, SEQ ID NO: 2. For example, a ratio of the peptide of SEQ ID NO: 1 to the peptide of SEQ ID NO: 2 ranges from about 1:10 to about 10:1, about 1:5 to about 5:1, about 1:4 to about 4:1, about 1:3 to about 3:1, or about 1:2 to about 2:1, or is about 1:1.

In some embodiments, the lipid particle is formed in-situ by combining simultaneously the lipid vesicle and the other components. In some embodiments, the lipid vesicle is formed and subsequently other components of the nanoparticle (nucleic acid components) are incorporated. In certain embodiments, the first nucleic acid and the conjugate are post-inserted to the liposome or lipid vesicle. In certain embodiments, a complex of the first nucleic acid and the conjugate is formed and subsequently inserted into the liposome or lipid nanoparticle by mixing.

Prior to forming the nanoparticle, the first nucleic acid is hybridized with the peptide to form a complex or a hybridized complex. The hybridization occurs between the complementary sequences between the first nucleic acid and the second nucleic acid when the peptide includes or is attached to the second nucleic acid. For example, the hydrogen bonding between two complementary nucleic acid bases may provide sufficient physical, stable conjugation.

In some embodiments, the first nucleic acid is not hybridized with the conjugate. In some embodiments, the unhybridized first nucleic acid is incorporated into the liposome. In certain embodiments, the nanoparticle may include unhybridized first nucleic acid as well as the hybridized first nucleic acid in a form of complex with the conjugate. In some embodiments, the first nucleic acid is encapsulated in the nanoparticle and a second nucleic acid is incorporated into the liposome.

In some embodiments, the peptide is incorporated into the liposome. In certain embodiments, the peptide is conjugated to the first nucleic acid. In certain embodiments, the peptide is conjugated to the second nucleic acid that is hybridized to the first nucleic acid.

After forming the nanoparticle, the complex (the conjugate hybridized with the first nucleic acid) is present on the surface of the lipid nanoparticle. In some embodiments, the first nucleic acid, which may be hybridized or unhybridized, is present on the surface of the lipid nanoparticle. In some embodiments, the peptide is present on the surface of the lipid nanoparticle. In some embodiments, a ratio between the unhybridized first nucleic acid and the complex ranges from 1:1 to 1:100. In some embodiments, a ratio between the unhybridized first nucleic acid and the complex ranges from 100:1 to 1:1. In certain embodiments, a ratio of the unhybridized first nucleic acid to the complex ranges from 5:1 to 1:5.

In some embodiments, a ratio of the first nucleic acid to the lipid nanoparticle is about 1000:1, 900:1, 800:1, 600:1, 500:1, 400:1, 300:1, 200:1, 100:1, 90:1, 80:1, 70:1, 60:1, 50:1, 40:1, 30:1, 20:1, or 10:1. In certain embodiments, the ratio of the first nucleic acid to the lipid nanoparticle is about 500:1, 400:1, 300:1, 200:1, or 100:1. However,

In some embodiments, a ratio of the conjugate to the lipid nanoparticle is about 1000:1, 900:1, 800:1, 600:1, 500:1, 400:1, 300:1, 200:1, 100:1, 90:1, 80:1, 70:1, 60:1, 50:1, 40:1, 30:1, 20:1, or 10:1. In certain embodiments, the ratio of the conjugate to the lipid nanoparticle is about 500:1, 400:1, 300:1, 200:1, or 100:1.

In some embodiments, a ratio of the peptide to the lipid nanoparticle is about 1000:1, 900:1, 800:1, 600:1, 500:1, 400:1, 300:1, 200:1, 100:1, 90:1, 80:1, 70:1, 60:1, 50:1, 40:1, 30:1, 20:1, or 10:1. In certain embodiments, the ratio of the peptide to the lipid nanoparticle is about 500:1, 400:1, 300:1, 200:1, or 100:1.

Also provided is a method of preparing an immunogenic or vaccine composition described herein. In some embodiments, the method includes steps of providing the lipid vesicle, the first nucleic acid, and the peptide; optionally forming a conjugate by attaching the peptide to the second nucleic acid; forming a complex of the first nucleic acid and the peptide or a hybridized complex of the first nucleic acid and the conjugate; and forming a nanoparticle by combining the lipid vesicle and the complex or the hybridized complex. For example, the complex may be encapsulated in the lipid vehicle to form a nanoparticle.

In certain embodiments, the method includes steps of: preparing the lipid nanoparticle the first nucleic acid, and the peptide; forming a nucleic acid-amphiphile complex by attaching the first nucleic acid to the one or more anchoring motifs; preparing a conjugate by attaching the peptide to the second nucleic acid; forming a hybridized complex of the first nucleic acid and the conjugate; encapsulating the nucleic acid-amphiphile complex in the lipid nanoparticle; and forming a nanoparticle by combining the lipid vesicle and the other components.

The above steps of preparing the lipid vesicle (lipid nanoparticles) and preparing the different conjugates may be performed simultaneously or subsequently, without bound to the order of process. In some embodiments, the lipid vesicle (lipid nanoparticles), the first nucleic acid, the second nucleic acid, and the peptide may be obtained by synthetic means, which may be meant by commercially and generally available methods.

In some embodiments, the conjugate may be formed by conjugating or attaching the first nucleic acid to the one or more lipid components or to the anchoring motifs (e.g., hydrophobic tail). In some embodiments, another conjugate may be formed by conjugating or attaching the peptide to the one or more lipid components or to the anchoring motifs (e.g., hydrophobic tail).

In some embodiments, a complex may be formed by the first nucleic acid and the peptide via direct attachment (e.g., chemical linkage) or indirect interaction (e.g., hydrophobic interaction). In some embodiments, the peptide is attached to the second nucleic acid, and a hybrid complex may be formed by hybridization between the complementary first nucleic acid and second nucleic acid.

The ratio between the peptide and the lipid nanoparticle is described above. In some embodiments, the conjugate may be formed by conjugating the peptide on the first nucleic acid-amphiphile. The ratio between the first nucleic acid and the lipid nanoparticle is described above. In some embodiments, the conjugate may be formed in an admixture of the peptide and the second nucleic acid. In some embodiments, the amount of peptide added to the admixture may be in excess compared to the amount of the second nucleic acid. In certain embodiments, a ratio of the peptide and the second nucleic acid in the admixture is about 10:1, 9:1, 8:1, 7:1, 6:1, 5:1, 4:1, 3:1, 2:1, and 1:1. In certain embodiments, a ratio of the peptide and the second nucleic acid in the admixture ranges from about 1:1 to about 4:1.

In some embodiments, the method may further include adding the first nucleic acid that is not hybridized with the conjugate. The ratio between the unhybridized first nucleic acid and the conjugate is described above.

The lipid nanoparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., WO 2013/082111, herein incorporated by reference in its entirety). Other physical features the geometrically engineering particles can include are, but are not limited to, fenestrations, angled arms, asymmetry and surface roughness, and charge that can alter the interactions with cells and tissues.

The lipid nanoparticle as described herein can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM) from the Institut fur Mikrotechnik Mainz GmbH, Mainz Germany. In some embodiments, methods of making lipid nanoparticle as described herein using SHM further comprise mixing at least two input streams wherein mixing occurs by microstructure-induced chaotic advection (MICA). According to this method, fluid streams flow through microchannels present in a herringbone pattern causing rotational flow and folding the fluids around each other. This method can also comprise a surface for fluid mixing wherein the surface changes orientations during fluid cycling. Methods of generating lipid nanoparticle as described herein using SHM include those disclosed in U.S. Pub. Nos. US20040262223 and US20120276209, each of which is incorporated herein by reference in their entirety.

In some embodiments, the immunogenic or vaccine composition may include a pharmaceutically acceptable excipient or a carrier. In some embodiments, the vaccine composition may further include a solvent, a stabilizer, a preservative, or combinations thereof. In addition, the vaccine composition may further include, but are not limited to, cell penetration agents, a delivery agent, a bioerodible or biocompatible polymer, and/or a sustained-release delivery depot. Types of pharmaceutically acceptable excipients may include any inactive ingredients known in the related art to the time of this disclosure, but not limited thereto.

### Methods of Treatment

In an aspect, provided is a method of treating or preventing a cancer in a subject. The method includes administering the immunogenic composition or vaccine composition as described herein.

In some embodiments, the cancer is a pancreatic cancer, or pancreatic adenocarcinoma. In some embodiments, the subject is administered with the vaccine composition as describe herein after diagnosed with the cancer. Alternatively, the subject does not have the cancer. In some embodiment, the subject is in moderate or considerable risk of cancer. For example, a risk of cancer may be determined by a physician, a doctor, or an expert based on factors including age, a personal or family history of cancer, use of tobacco, obesity, alcohol, types of viral infections, exposure to chemicals or radiation, or the like. In some embodiments, the subject does not have the cancer or is in insignificant risk of cancer.

The immunogenic compositions and vaccine compositions described above can be administered by any route that results in a therapeutically effective outcome. These include, but are not limited to oral, pulmonary, rectal, parenteral, transdermal, subcutaneous, intravenous, intramuscular, intraperitoneal, inhalational, buccal, sublingual, intrapleural, intrathecal, intranasal, and the like. Examples of routes of administration include parenteral, e.g., intravenous, intradermal, subcutaneous, oral (e.g., inhalation), transdermal (topical), and transmucosal administration. In some embodiments, compositions can be administered in a way that allows them to cross the blood-brain barrier, vascular barrier, or other epithelial barrier. In certain embodiments, the vaccine composition is administered by subcutaneous or intramuscular injection.

In some embodiments, a formulation for the administration route above can include at least one inactive ingredient.

Injectable formulations can be sterilized, for example, by filtration through a bacterial-retaining filter, and/or by incorporating sterilizing agents in the form of sterile solid compositions that can be dissolved or dispersed in sterile water or other sterile injectable medium prior to use.

Injectable formulations can be for direct injection into a region of a tissue, organ and/or subject. As a non-limiting example, a tissue, organ and/or subject can be directly injected a formulation by intramyocardial injection into the ischemic region. (See, e.g., Zangi et al. Nature Biotechnology 2013, which is herein incorporated by reference in its entirety).

In order to prolong the effect of an active ingredient, it is often desirable to slow the absorption of the active ingredient from subcutaneous or intramuscular injection. In some embodiments, this type of administration can be accomplished by the use of a liquid suspension of crystalline, highly hydrophobic or amorphous material with poor water solubility.

Dosages of the vaccine composition may be varied depending upon the requirements of the patient and the immunogenic or vaccine composition being employed. The dose administered to a patient, in the context of the disclosure should be sufficient to effect a beneficial therapeutic and preventive effects (e.g., immune response) in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the vaccine composition. Thereafter, the dosage is increased by small increments until the optimum effect under circumstances is reached. Dosage amounts and intervals can be adjusted individually to provide levels of the administered vaccine effective for the particular clinical indication being treated. This will provide a therapeutic regimen that is commensurate with the severity of the individual's disease state.

Also provided is a kit including the vaccine composition as described herein. The kit may further include an applicator (e.g., injector) and/or a product instruction.

### EXAMPLES

### Example 1: Preparation of Lipid Nanoparticles

Peptide-based cancer vaccines typically consist of a sequence of amino acids derived from tumor-specific antigens. There have been reported synthetic peptides that can induce vaccine-specific T cells within the tumor in a murine pancreatic cancer model. For example, in the related art, twenty-amino acid-long peptides with mutated residues at position 11 have shown that these peptides can promote a strong antitumor immunity (Kinkead et al., JCI Insight. 2018).

### Methods

### Liposome Synthesis

Liposomes composed of 99 mol % DPPC and 1 mol % DSPE-PEG750 were generated using the thin-film technique. Briefly, lipids were combined at the designated ratio in chloroform at a total lipid concentration of 20 mM. A thin film was generated via rotary evaporation and overnight drying under vacuum. The thin film was rehydrated in PBS under rotation, and the liposomes were extruded via 21 passages through a 100 nm PC membrane. These liposomes were then analyzed for their size via dynamic light scattering (DLS) and their zeta potential (Zetasizer Nano ZS, Malvern Panalytical).

In the design where the CpG was encapsulated in the liposomes (FIGS. 12 D, E), 2 mL of 20 mM total lipids, 59% DPPC 40% cholesterol 1% 18:0 PEG750-PE were used. The added cholesterol was to try and prevent loss of encapsulated material during the post-insertion process. After the thin film was generated, it was rehydrated at 55° C. with rotation with 2 mL of a 1×HEPES buffered saline solution (1 g/L glucose, 5 g/L HEPES, 0.37 g/L KCl, 8 g/L NaCl, and 0.135 g/L Na₂HPO₄·2H₂O pH 7.2) that contained 300 nmol CpG. After rehydration this solution was subjected to 2 freeze-anneal-thaws in which it was placed in liquid nitrogen for 3 min, allowed to anneal in ice water for 30 min, and then thawed at 55° C. for 3 min. Given that no cationic lipids were used, this would be expected to have relatively low encapsulation, so it was subjected to diafiltration into 1×PBS using a 100 kDa MWCO Amicon centrifugal filter. The RiboGreen assay was used to determine how much CpG had been encapsulated. This entire sample was combined with 60 nmol peptide amphiphiles and shaken at 400 rpm and 37° C. overnight to allow for post-insertion. These post-inserted liposomes were purified via diafiltration against 1×PBS with a 100 kDa MWCO Amicon centrifugal filter to remove unincorporated peptide amphiphiles and CpG. The final liposomes were characterized by DLS/Zeta to determine size and charge, BCA to determine the final peptide concentration, and RiboGreen to determine the final CpG concentration.

In the design where the CpG was encapsulated in the nanoparticle as shown in FIG. 12F, 3.69 mL of 50 mM sodium acetate pH 4.0 aqueous solution containing 75 nmol of CpG was generated. In a separate tube, combined 674 μL 10 mg/mL D-Lin-MC3-DMA, 165.9 μL 10 mg/mL DSPC, 79.0 μL 10 mg/mL PEG2k-DMG, and 312.6 μL 10 mg/mL cholesterol for a total volume of 1.23 mL and an aqueous to ethanol ratio of 3:1. All stocks were in 100% ethanol. This represents a 5:1 N:P ratio and a 50:10:1.5:38.5 molar ratio of the components in the order listed. Both the aqueous and ethanol solutions were loaded into syringes and combined using a Fluidic 187 herringbone mixer microfluidic chip (microfluidic ChipShop, Jena, Germany). The aqueous solution was pumped at 3 mL/min and the ethanol solution was pumped at 1 mL/min. The collected LNP product was dialyzed against 1×PBS using a 3500 Da MWCO Pur-A-Lyzer Maxi Dialysis kit. After dialysis these LNPs were 0.2 μm PES sterile filtered. This entire sample was combined with 60 nmol peptide amphiphiles and shaken at 400 rpm and 37° C. overnight to allow for post-insertion. These post-inserted liposomes were purified via diafiltration against 1×PBS with a 100 kDa MWCO Amicon centrifugal filter to remove unincorporated peptide amphiphiles and CpG. The final liposomes were characterized by DLS/Zeta to determine size and charge, BCA to determine the final peptide concentration, and RiboGreen to determine the final CpG concentration.

### SEQUENCE LISTING

TABLE 1
peptides

| SEQ ID NO. | Amino Acid Sequence | Gene Symbol |
|---|---|---|
| SEQ ID NO: 1 | GSSAEESHLSCLNWSTLVPL | GIBI12 |
| (peptide 44) | | |
| SEQ ID NO: 2 | IKIVRLTTGSAYQFRVCAEN | Tnt |
| (peptide 237) | | |

Underlined amino acid represents mutated residue.

TABLE 2
nucleic acids

| SEQ ID NO. | Nucleic acid sequence | |
|---|---|---|
| SEQ ID NO: 3 | 5′-AACGTCAGGAACGTCATGGA⁺-3′ | ssDNA sequence |
| | | used for peptide SEQ |
| | | ID NOs: 1-2 |
| SEQ ID NO: 4 | 5′-⁺AACGTCAGGAACGTCATGGA-3′ | ssDNA sequence |
| | | used for peptide SEQ |
| | | ID NO: 2 |
| SEQ ID NO: 5 | 5′-T*C*C*A*T*G*A*C*G*T*T*C*C*T*G*A*C* | CpG 1826 murine |
| | G*T*T*T*T*T*T*T*T*T*T*⁺⁺-3′ | sequence |
| SEQ ID NO: 6 | 5′-T*C*G*TC*G*T*T*T*T*G*T*C*G*T*T*T*T | CpG 2006 (also |
| | G*T*C*G*T*T*T*T*T*T*T*T*T*T*⁺⁺-3′ | known as CpG 7909 |
| | | for humans) |
| SEQ ID NO: 7 | 5′-T*C*G*T*C*G*T*T*T*T*G*T*C*G*T*T*T* | CpG 2006-21798 |
| | T*G*T*C*G*T*T*T*CG*T*CG*A*CG*A*T*CG* | |
| | G*C*G*CG*C*G*C*C*G-TT TT TT TT*⁺⁺-3′ | |
| SEQ ID NO: 8 | T*T*T*T*T⁺⁺-3′ | 20 poly-T |

⁺represents thiol modifier at 5′ or 3′ end of the oligonucleotides.
*represents phosphorothioate linkages
⁺⁺represents amino group at modification at 3′ end of the nucleotide as linker

### CpG-Amphiphile Synthesis and Nucleic Acid-Amphiphile Synthesis

Dialkyl (e.g., C₁₆ alkyl) tails were synthesized. Two 1-hexadecanol were conjugated to the carboxylic acid groups of an L-glutamic acid through ester linkages. A succinic acid linker was reacted with the glutamic acid amino group to form an amide bond. The terminal carboxylic acid group was then NHS activated to complete the tail synthesis.

The CpG sequences having SEQ ID NO: 5, SEQ ID NO: 6, SEQ ID NO: 7, and nucleic acid sequence having SEQ ID NO: 8 was purchased from Integrated DNA Technology. This conjugation of the tail to the CpG or nucleic acid sequence was performed as follows. The CpG or nucleic acid sequence was precipitated out of aqueous solution using cetyltrimethylammonium bromide. It was then dissolved in a 9:1 DMF:DMSO and reacted overnight at a temperature of 65° C. with 10× molar excess of the dialkyl tail. Most of the solvent was then removed via vacuum oven, and the amphiphiles were isolated using 2.5% w/v lithium perchlorate in acetone. The pelleted amphiphiles were then solubilized in MilliQ water and purified via reverse phase high performance liquid chromatography (HPLC) using a gradient of 100 mM hexafluoroisopropanol 15 mM triethylamine in MilliQ and 100 mM hexafluoroisopropanol 15 mM triethylamine in methanol.

ssDNA-Peptide Synthesis

Oligonucleotides (SEQ ID NOs: 3-4) were ordered from Integrated DNA Technology. These oligonucleotides were reduced via incubation with either 100× molar excess TCEP (tris(2-carboxyethyl)phosphine hydrochloride) or 500 mM DTT (dithiothreitol). The reducing agent was removed and replaced with MilliQ water via diafiltration utilizing 3 kDa MWCO Amicon centrif μgal filters. The thiols were then activated using 50 mM 2-aldrithiol. Excess aldrithiol was removed and buffer exchanged with MilliQ water using 3 kDa MWCO Amicon centrifugal filters and the activated ssDNA was quantified using absorbance at 260 nm. For the peptide of SEQ ID NO: 1, the activated ssDNA was combined with a 5× molar excess of peptide of SEQ ID NO: 1 in a 100 mM sodium phosphate pH 8.0 solution and reacted overnight at 50° C. For the peptide of SEQ ID NO: 2, the activated ssDNA was combined at a 1:1 molar ratio with peptide 237 in a 100 mM sodium phosphate pH 8.0 solution which had been supplemented with DMF (10% v/v) and 2 M TEAA pH 7.0 (10% v/v) to help solubilize the peptide. After reacting overnight at a temperature of 50° C., the peptide of SEQ ID NO: 4 was added to a final molar ratio of 1:2 ssDNA: peptide along with another 2 M TEAA and DMF in the same volumes added on the previous day. Solutions were centrifuged to pellet out unreacted peptide, diluted in MilliQ water, and purified via reverse phase HPLC using a gradient of 100 mM hexafluoroisopropanol 15 mM triethylamine in MilliQ water and 100 mM hexafluoroisopropanol 15 mM triethylamine in methanol. These samples were then concentrated by airflow and characterized by absorbance at 260 nm to measure ssDNA recovery, and thro μgh BCA analysis to evaluate peptide recovery. BCA analysis always utilized a standard curve generated from the corresponding peptide of interest.

ssDNA Hybridization

The ssDNA-peptide conjugates were then hybridized to the CpG-amphiphiles by combining them in a 1:1 molar ratio, spiking them with 10× duplex buffer stock to a final concentration of 1× duplex buffer (30 mM HEPES, 100 mM potassium acetate pH 7.5, and heating them to a temperature of 70° C. for 10 min before allowing them to cool to room temperature over several hours. These hybridized ssDNA-peptide/CpG-amphiphile conjugates were then post-inserted into the liposomes by combining them at a 400:1 peptide-CPG conjugate: liposome molar ratio (moles peptide-CPG conjugate: moles liposomes) in 1×PBS and shaking them at a temperature of 37° C. overnight. For later experiments additional non-hybridized CpG-amphiphiles were also added during the post-insertion process such that the molar ratio was 400:400:1 CpG-amphiphiles:peptide-CPG conjugate: liposomes in order to enhance the immunostimulatory effect. The post-inserted liposomes were purified from free ssDNA-peptide and CpG-amphiphiles via Amicon 100 kDa MWCO centrifugal filters using repeated diafiltration into 1×PBS. They were then concentrated to the final targeted concentration using these centrifugal filters and analyzed for their size via DLS and their zeta potential (Zetasizer Nano ZS, Malvern Panalytical). The final peptide concentration was determined via BCA assay using the corresponding peptides to generate the standard curve.

Successful post-insertion of CpG-amphiphiles into liposomes was verified with a gel assay. For that, liposomes were combined with the CpG-amphiphiles at 200:1, 400:1, 600:1, and 800:1 molar ratios of CpG-amphiphiles:liposomes and post-inserted at 37° C. overnight with shaking. These samples were then loaded onto a 1% Agarose gel and run at 120 V for 35 min. The CpG-amphiphiles were then stained using Diamond Nucleic Acid dye (Promega) and visualized on a ChemiDoc MP Imaging System (Bio-rad).

### Peptide-Amphiphile Synthesis and Incorporation in the Lipid Nanoparticles

The peptide (in DMSO) was combined with 3-5 molar excess dialkyl C₁₆ tail in DMF. 10 μL TEA (~75×molar excess) was added and the solution was reacted at 40° C. with stirring for 2-4 hours. After the incubation, the reaction was diluted in MilliQ water to crash out unreacted tail and the reaction was quenched by spiking in 1M Tris pH 8. The solution was then centrifuged to pellet the unreacted tail and the supernatant was isolated and purified via reverse phase HPLC using a C₃ column and a gradient of Water+HFIP+TEA to MeOH+HFIP+TEA.

Peptide-amphiphiles were incorporated in the lipid nanoparticles either by mixing with other lipid components or via post-insertion as described above. The final peptide concentration was determined via the BCA assay as discussed above.

For designs that used peptide amphiphiles and CpG amphiphiles, peptide amphiphiles and CpG amphiphiles at a 400:400:1 ratio of peptide amphiphiles:CpG amphiphiles:liposomes were shaken at 400 rpm and 37° C. overnight to allow for post-insertion. These post-inserted liposomes were purified via diafiltration against 1×PBS with a 100 kDa MWCO Amicon centrifugal filter to removed unincorporated peptide amphiphiles and CpG amphiphiles. The final liposomes were characterized by DLS/Zeta to determine size and charge, BCA to determine the final peptide concentration, and RiboGreen to determine the final CpG concentration.

Peptide Conjugation to Nucleic Acid-Amphiphile and their Incorporation in the Lipid Nanoparticles

Peptides were conjugated to nucleic acid-amphiphiles as described above in ssDNA-peptide synthesis.

The resulting compounds were incorporated in the lipid nanoparticles via post-insertion as described above. The final peptide concentration was determined via the BCA assay as discussed above.

### Evaluation of Molar Liposome Concentration

The total lipid concentration was determined utilizing the Stewart assay (Hein et al., Org. Biomol. Chem., 2016), The size was analyzed by DLS using a Zetasizer NanoZS (Malvern Panalytical). The size could be converted to lipids per liposome using the following equation:

$${N}_{tot} = 17.69 [ {( \frac{d}{2} )}^{2} + {( \frac{d}{2} - 5 )}^{2} ]$$

Where d is the diameter of the liposome and N is the number of lipids per liposome. The concentration of lipids determined by the Stewart assay could then be divided by the total number of lipids per liposome to give the concentration of liposomes.

### Results

Ratios describe ratios of CpG-amphiphiles to individual liposomes.

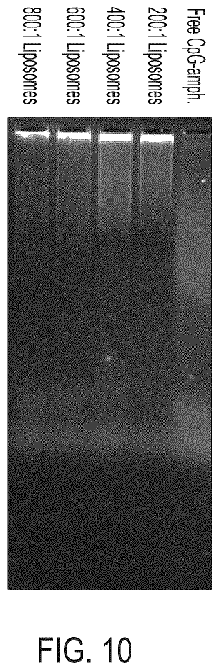

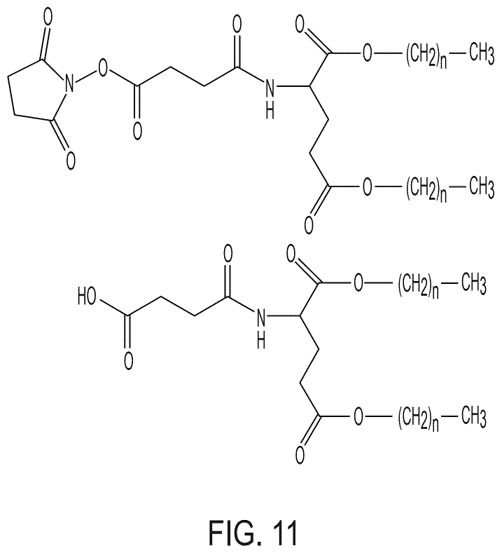

As shown in FIG. 10, a large majority of the CpG-amphiphiles were successfully post-inserted at all ratios, as indicated by the strong band present at the top of each, not present in the free CpG-amphiphile lane. The following Table 3 shows charge and size measured for liposomes, CpG Liposomes, 44-CpG Liposomes, and 237-CpG Liposomes that were measured as described herein.

TABLE 3
Size and charge of different liposomal formulations

| Sample | Zeta Potential (mV) | Z avg. (nm) | Peak 1 Size (nm) | PDI |
|---|---|---|---|---|
| Liposomes | −9.9 ± 2.1 | 109.1 ± 1.2 | 113.0 ± 2.5 | 0.06 ± 0.04 |
| CpG Liposomes | −45.0 ± 2.2 | 122.7 ± 1.3 | 123.9 ± 3.2 | 0.11 ± 0.11 |
| 44-CpG Liposomes | −38.7 ± 2.8 | 127.0 ± 2.7 | 130.5 ± 2.4 | 0.06 ± 0.03 |
| 237-CpG Liposomes | −38.7 ± 8.0 | 121.4 ± 2.5 | 124.5 ± 1.8 | 0.09 ± 0.04 |

### Example 2: LNP-CpG Peptide Vaccine Preclinical Data

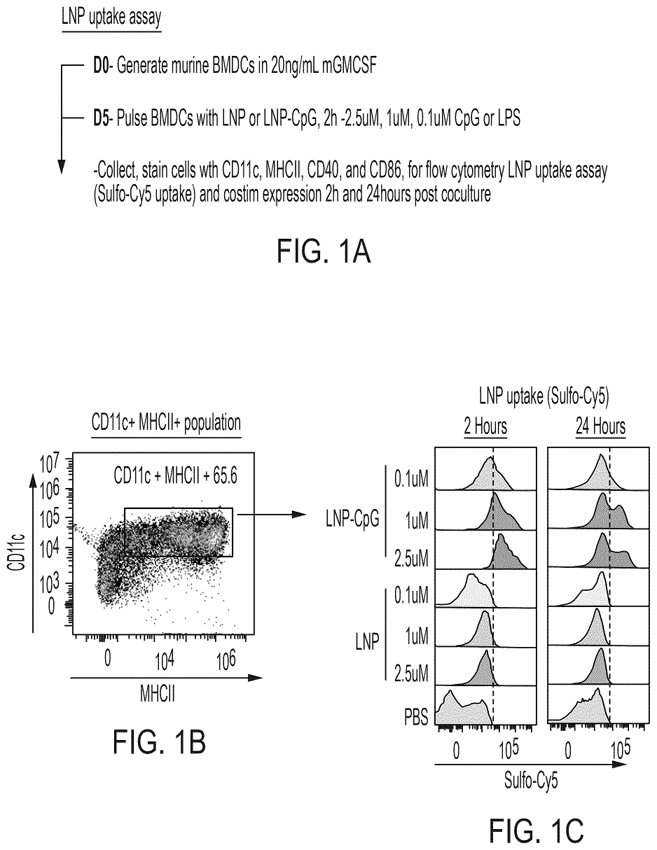

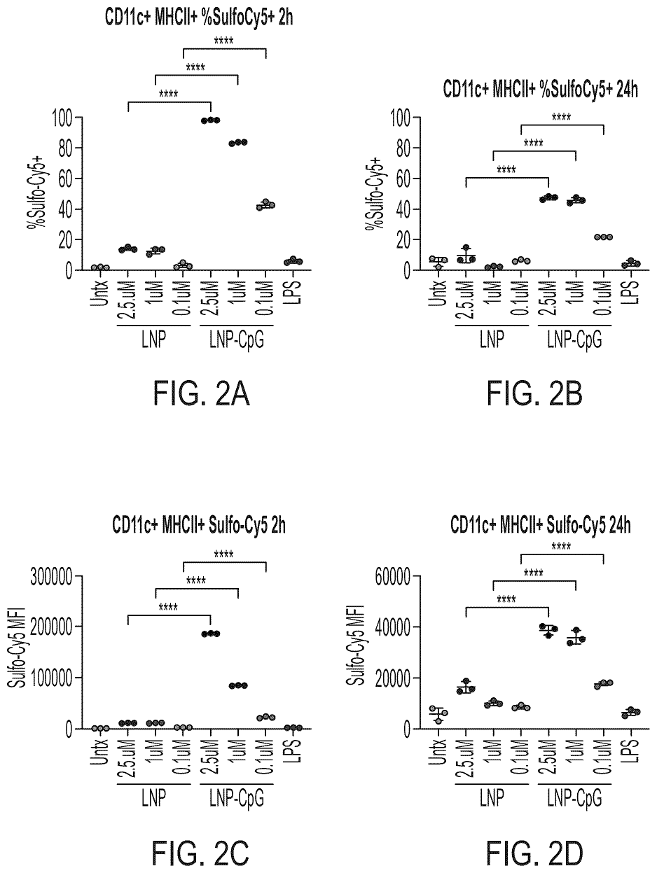

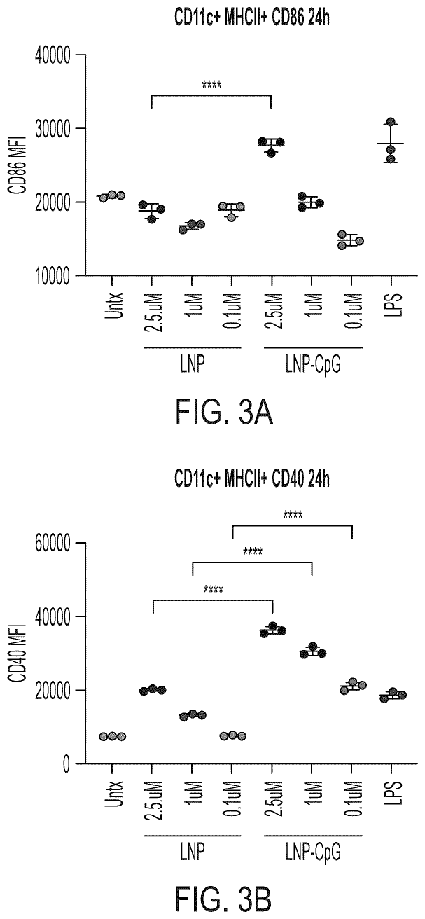

As shown in FIG. 1A, uptake assay of CpG decorated lipid nanoparticles (LNP), LNP-CpG, was performed with murine antigen presenting cells, (murine bone marrow derived dendritic cells, BMDCs). On day 0, bone marrow from C57BL/6 mice was isolated and cultured with 20 ng/mL mGMCSF for 5 days. BMDCs were then collected and cultured for 2 hours with 2.5 μM, 1 μM, or 0.1 μM non-decorated LNP or LNP-CpG encapsulating sulfo-Cy5 dye. Nanoparticles were then removed and cells were washed with PBS. LNP-pulsed cells were collected immediately after wash or 24 hours later and analyzed by flow cytometry for sulfo-cy5 dye uptake in dendritic cells (CD11c, MHCII) with activation markers (CD86, CD40). As shown in FIG. 1B-1C, LNP-CpG had improved uptake by BMDCs.

In addition, BMDCs were likely to preferentially uptake LNP-CpG as indicated by higher percent of sulfo-Cy5 labeled cells and median fluorescent intensity (MFI) increase in sulfo-cy5 stained cells. LNP-CpG significantly enhanced BMDC activation and costimulatory molecule expression in vitro as well.

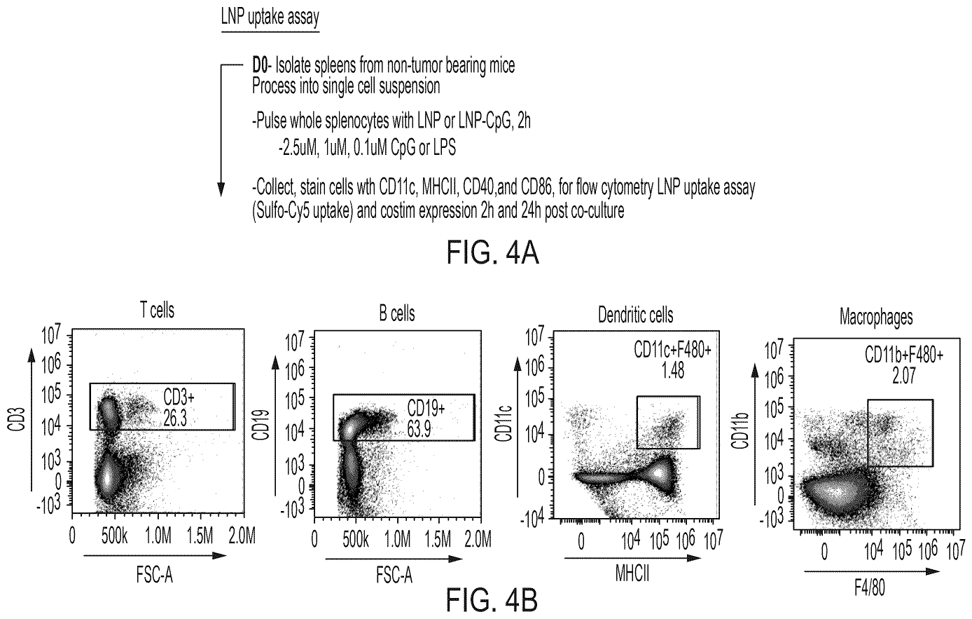

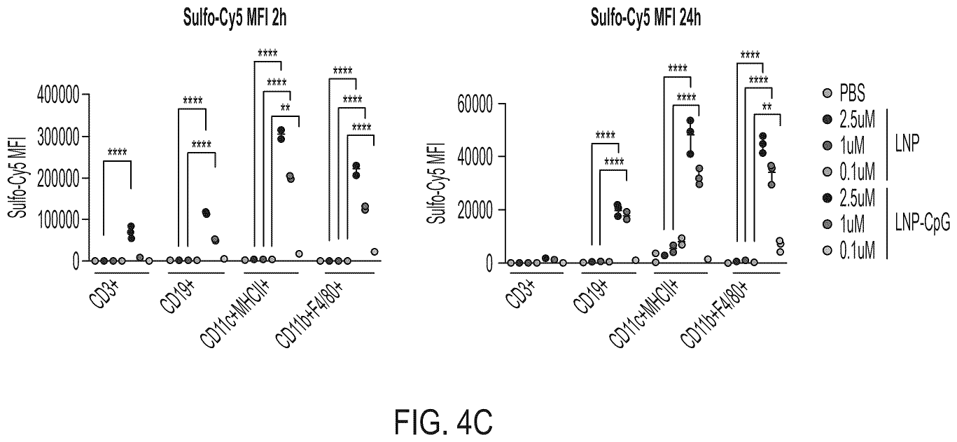

As shown in FIG. 4A, uptake assay of LNP-CpG was performed with murine splenocytes. On day 0, mouse spleens were isolated and processed to single cell suspension. Whole splenocytes were cultured for 2 hours with 2.5 μM, 1 μM, or 0.1 μM non-decorated LNP or CpG decorated LNP (LNP-CpG) encapsulating sulfo-Cy5. Nanoparticles were then removed and cells were washed with PBS. LNP-pulsed cells were collected immediately after wash or 24 hours later and analyzed by flow cytometry for sulfo-cy5 dye uptake. As shown in FIG. 4C, LNP-CpG were taken up mainly by dendritic cells and macrophages, but also B cells and T cells, and their cell association was significantly higher compared to non-CpG nanoparticles (LNP).

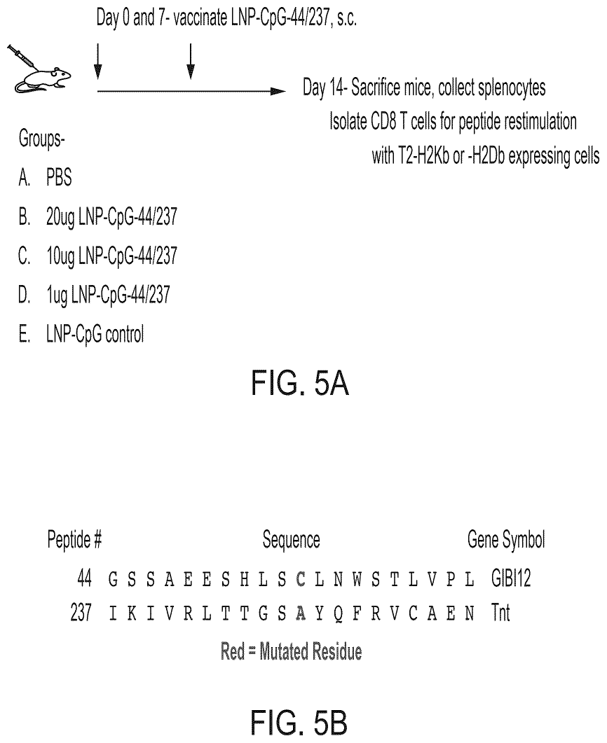

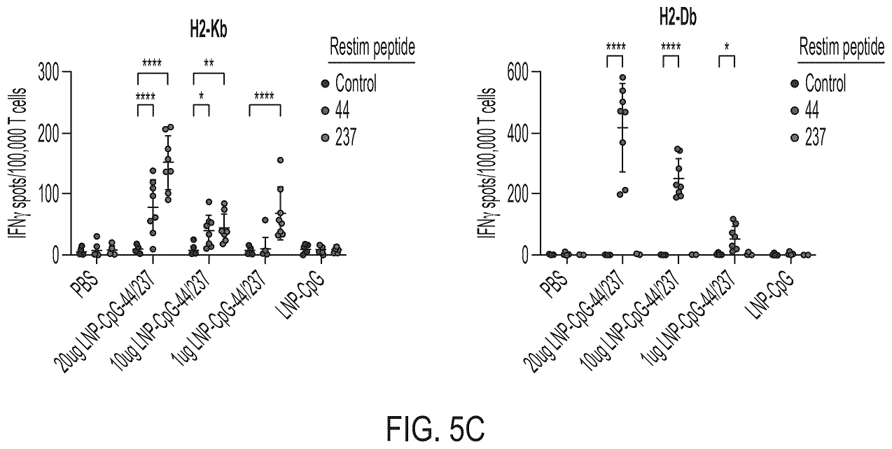

As shown in FIG. 5A, in vivo immunogenicity testing of CpG decorated LNP (LNP-CpG) carrying murine pancreatic cancer line Panc02 neoantigens 44 and 237 was performed. Mice were vaccinated on day 0 and day 7 with PBS control, LNP-CpG control, or LNP-CpG-44/237 at 20 μg (10 μg/peptide), 10 μg (5 μg/peptide) and 1 μg (0.5 μg/peptide). On day 14, spleens were collected from vaccinated mice and used for ELISPOT. As shown in FIG. 5C and FIG. 13 (peptide 44 was evaluated), LNP-CpG-44/237 peptide vaccine induced robust epitope-specific T cell responses in vivo, in view of IFNγ production by CD8+ T cells from vaccinated mice after overnight incubation with T2-H2-Kb and T2-H2-Db antigen presenting cells pulsed with control peptide.

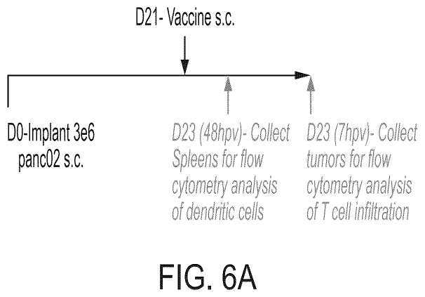

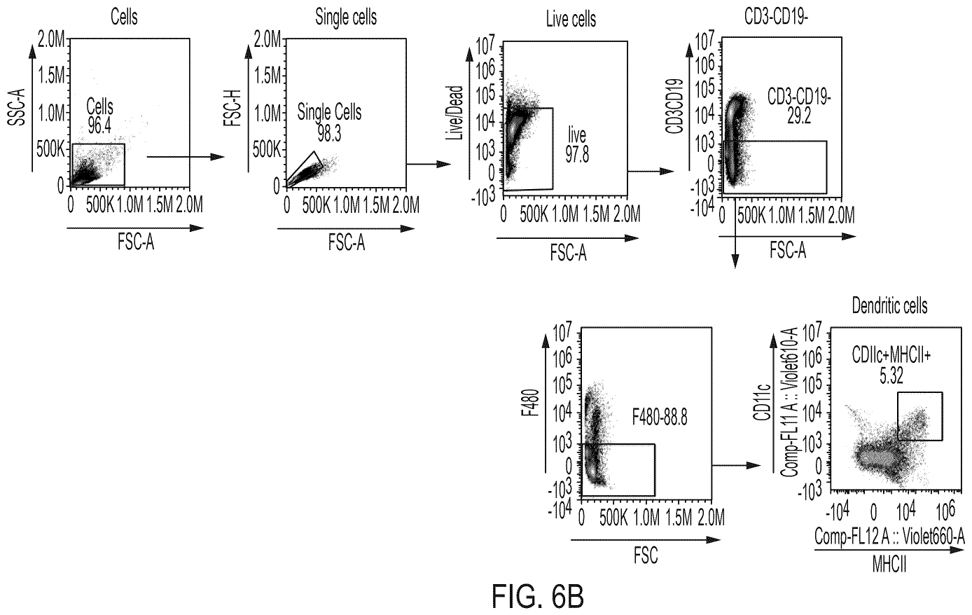

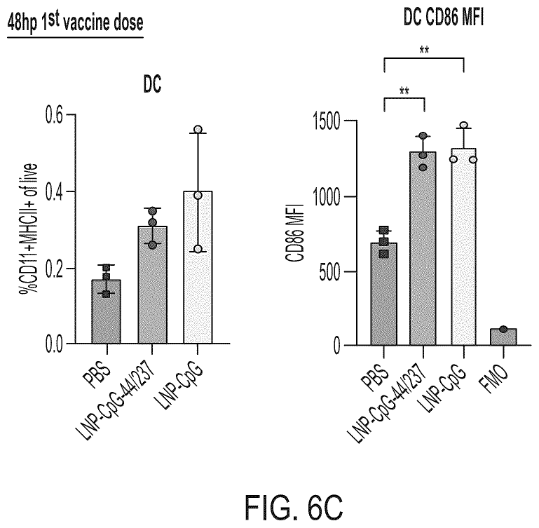

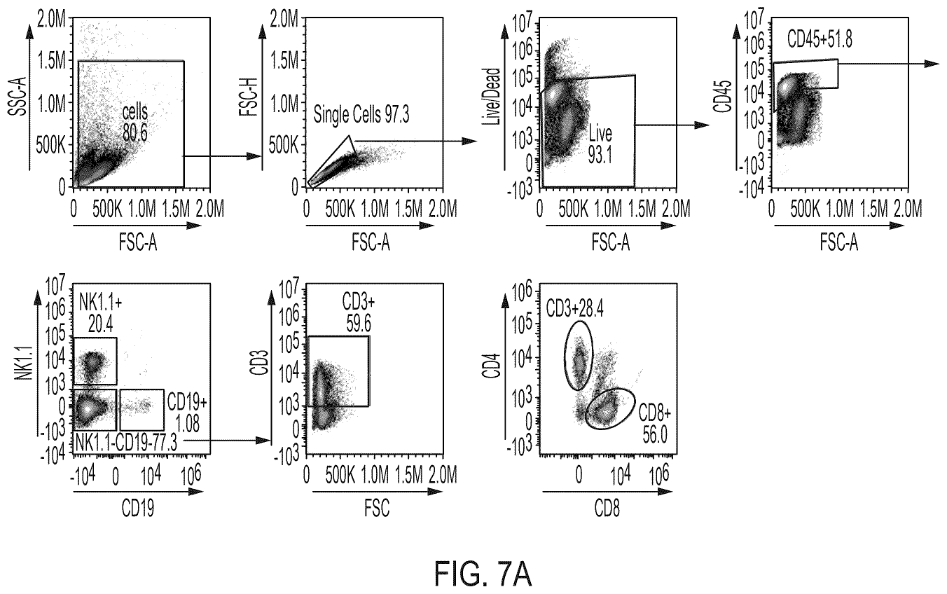

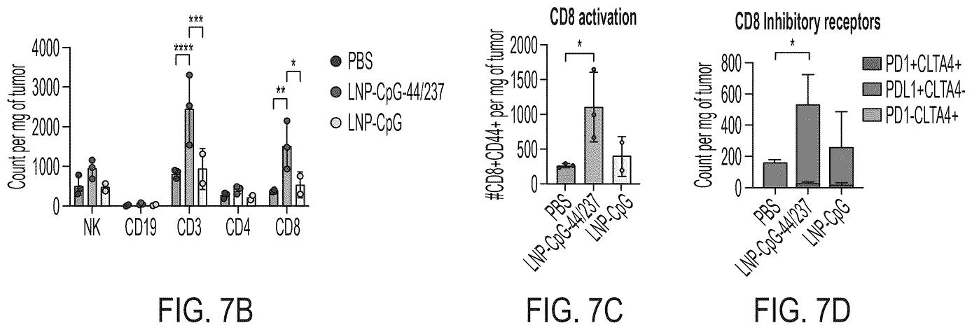

As shown in FIG. 6A, in vivo immunogenicity testing of CpG decorated LNP (LNP-CpG) carrying murine pancreatic cancer line Panc02 neoantigens 44 and 237 was performed. Mice were implanted with 3×10⁶ Panc02 cells, subcutaneously. 21 days later, mice were vaccinated with PBS control, LNP-CpG, or LNP-CpG-44/237 (5 μg/peptide), subcutaneously. Splenocytes and tumors were collected 48 hours or 7 days post vaccination and analyzed by flow cytometry for myeloid and lymphoid cell activation. As results show, splenic dendritic cells expressed significantly more costimulatory molecules 48 hours after LNP-CpG vaccination (FIG. 6C). Indeed, LNP-CpG-44/237 peptide vaccination resulted in significantly more activated CD8+ T cell infiltration in the tumor compared to LNP-CpG control as shown in FIGS. 7B-7D.

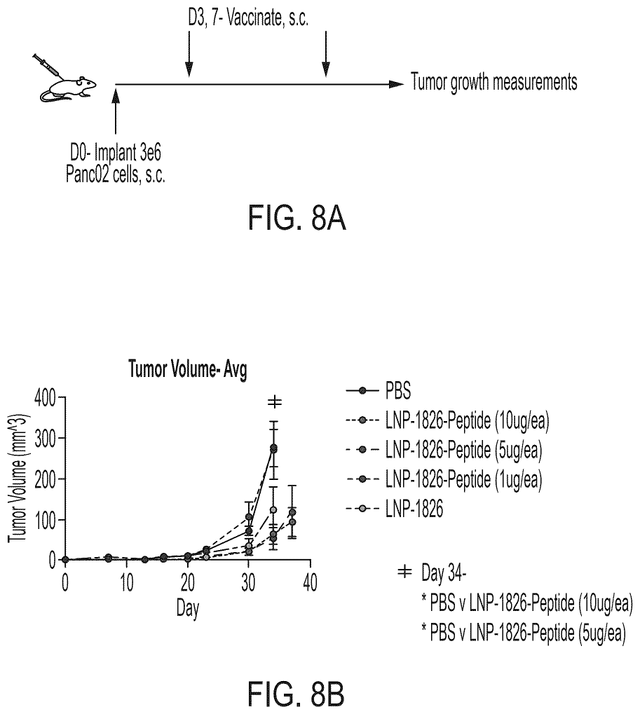

As shown in FIG. 8A, therapeutic efficacy analysis of LNP-CpG-44/237 vaccine in the murine panc02 model was performed. Mice were implanted with 3×10⁶ panc02 cells, subcutaneously. On day 3 and day 7, mice receive PBS, LNP-CpG control, or LNP-CpG-44/237 vaccine at a dose of 10 μg, 5 μg, or 1 μg per peptide. Tumors were monitored for growth. LNP-CpG-peptide vaccine significantly improved control of tumor growth in FIG. 8B.

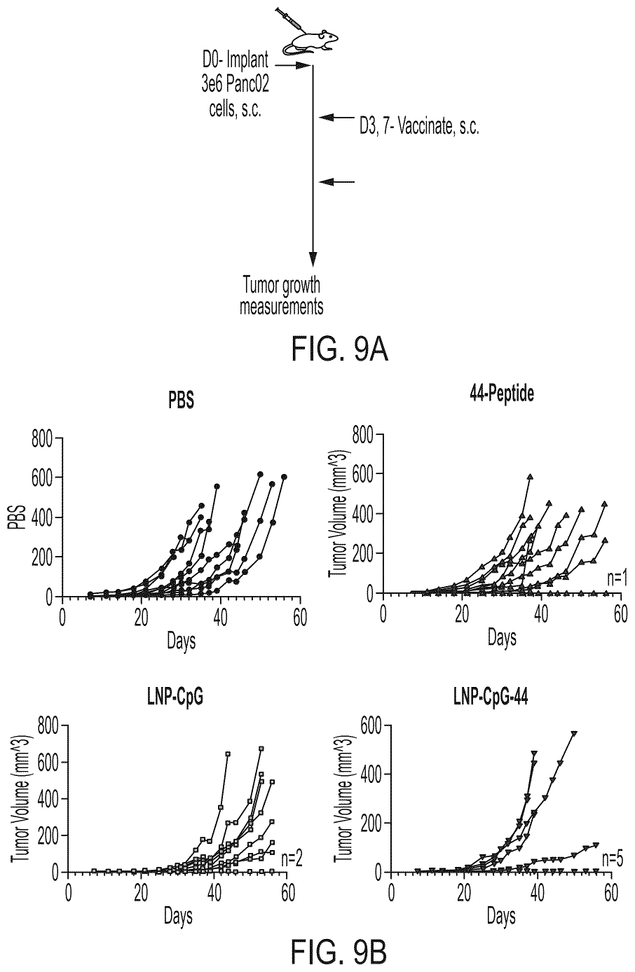

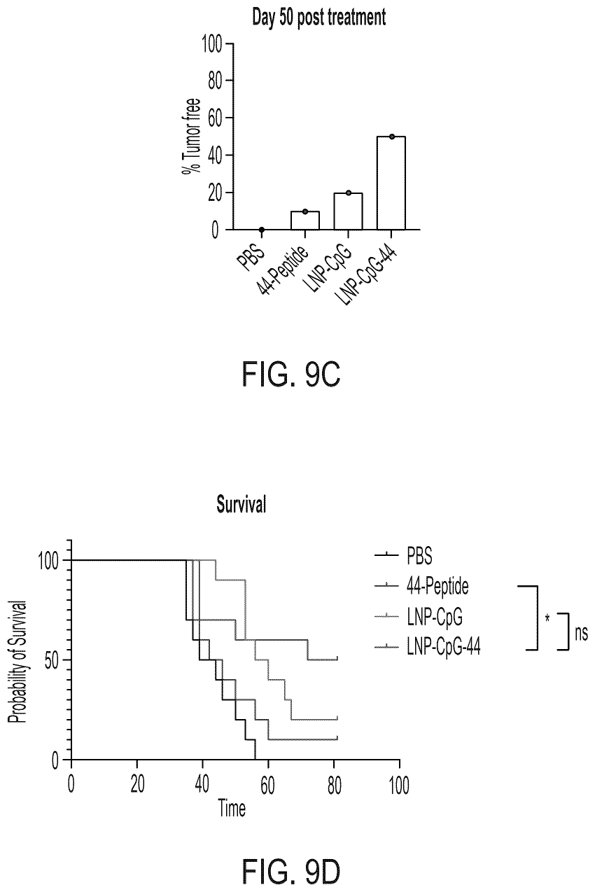

As shown in FIG. 9A, therapeutic efficacy analysis of LNP-CpG-44 vaccine in the murine panc02 model was performed as compared to peptide vaccine or LNP-CpG control nanoparticle. Mice were implanted with 3×10⁶ panc02 cells, subcutaneously. On day 3 and day 7, mice receive PBS, 44-peptide based vaccine (Addavax+ADUS100 agonist), LNP-CpG control, or LNP-CpG-44 (10 μg/peptide). As shown in FIGS. 9B-9C, LNP-CpG-peptide vaccine removed tumors in 5/10 mice treated.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the disclosure will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. An immunogenic composition comprising:
a) a lipid vesicle comprising one or more lipid components;
b) a first nucleic acid;
c) a peptide; and
d) one or more anchoring motifs containing one or more hydrophobic tails,
wherein the first nucleic acid forms a conjugate with the one or more anchoring motifs.

2. The immunogenic composition of claim 1, wherein the one or more hydrophobic tails comprise C₅-C₃₀ alkyl group, C₅-C₃₀ alkenyl groups, or combinations thereof.

3. The immunogenic composition of claim 1, wherein the peptide is conjugated to the one or more the anchor compounds.

4. The immunogenic composition of claim 1, wherein the peptide is conjugated to the one or more lipid components.

5. The immunogenic composition of claim 1, wherein the peptide is conjugated to the first nucleic acid.

6. The immunogenic composition of claim 5, wherein the peptide further comprises a second nucleic acid that has a sequence complementary to a sequence of the first nucleic acid.

7-9. (canceled)

10. The immunogenic composition of claim 1, wherein the one or more lipid components comprises phospholipids and are functionalized with a functional group, a small molecule, polymer, a peptide, a ligand, or combinations thereof.

11. The immunogenic composition of claim 1, wherein the one or more lipid components comprise one or more selected from the group consisting of polyethylene glycol (PEG); dipalmitoylphosphatidylcholine (DPPC); distearoylphosphatidylcholine (DSPC); DPPE; 1,2-distearoyl-sn-glycero-3-phosphoethanolamine (DSPE), DSPE-PEG amine; 1,2-dipalmitoyl-sn-glycero-3-phosphoethanolamine-N-[4-(p-maleimidomethyl)cyclohexane-carboxamide]; 1,2-dioleoyl-sn-glycero-3-phosphoethanolamine-N-[4-(p-maleimidomethyl)cyclohexane-carboxamide]; 1,2-distearoyl-sn-glycero-3-phosphocholine (N-aminoethyl); 1,2-dioleoyl-sn-glycero-3-phosphocholine (N-aminoethyl); 1,2-dioleoyl-sn-glycero-3-phosphoethanolamine-N-[4-(p-maleimidophenyl)butyramide]; 1,2-dipalmitoyl-sn-glycero-3-phosphoethanolamine-N-[4-(p-maleimidophenyl)butyramide]; 1,2-dioleoyl-sn-glycero-3-phosphoethanolamine-N-(hexanoylamine); 1,2-dipalmitoyl-sn-glycero-3-Phosphoethanolamine-N-(hexanoylamine); 1,2-dipalmitoyl-sn-glycero-3-phosphothioethanol; 1,2-dioleoyl-sn-glycero-3-phosphoethanolamine-N-[3-(2-pyridyldithio)propionate]; 1,2-dipalmitoyl-sn-glycero-3-phosphoethanolamine-N-[3-(2-pyridyldithio)propionate]; 1,2-dioleoyl-sn-glycero-3-phosphoethanolamine-N-(succinyl); 1,2-dipalmitoyl-sn-glycero-3-phosphoethanolamine-N-(succinyl); 1,2-dioleoyl-sn-glycero-3-phosphoethanolamine-N-(glutaryl); 1,2-dipalmitoyl-sn-glycero-3-phosphoethanolamine-N-(glutaryl); 1,2-dioleoyl-sn-glycero-3-phosphoethanolamine-N-(dodecanoyl); 1,2-dioleoyl-sn-glycero-3-phosphoethanolamine-N-(cap biotinyl); 1,2-dipalmitoyl-sn-glycero-3-phosphoethanolamine-N-(cap biotinyl); 1,2-dipalmitoyl-sn-glycero-3-phosphoethanolamine-N-(cyanur); 1,2-dipalmitoyl-sn-glycero-3-phosphoethanolamine-N-(6-((folate)amino)hexanoyl); 1,2-dipalmitoyl-sn-glycero-3-phospho((ethyl-1′,2′,3′-triazole)triethyleneglycolmannose); 1,2-distearoyl-sn-glycero-3-phosphoethanolamine-N-square; 1,2-dioleoyl-sn-glycero-3-phospho(ethylene glycol); 1,2-dipalmitoyl-sn-glycero-3-phospho(ethylene glycol); and 1,2-distearoyl-sn-glycero-3-phosphoethanolamine-N-[(polyethylene glycol)-2000]-N-[4-(triphenylphosphonium)butanamide]; DMG-PEG, DSG-PEG, DOPE-PEG, DOPE-PEG-amine, DOPE-PEG-COOH, DOPE-PEG-Mal, M-PEG-DMPE, M-PEG-DOPE, DSPE-PEG, DSPE-PEG-Amine, DSPE-PEG-Acid, DSPE-PEG-COOH, DSPE-PEG-CH₂COOH, DSPE-PEG-NHS, DSPE-PEG-Azide, DSPE-PEG-DBCO, DSPE-PEG-Aldehyde, DSPE-PEG-Biotin, DSPE-PEG-Folate, DSPE-PEG-OH, DSPE-PEG-SH, DSPE-PEG-SPDP, DSPE-PEG-IA, DSPE-PEG-Vinylsulfone, DSPE-PEG-Cy3, DSPE-PEG-Cy5, DSPE-PEG-FITC, DSPE-PEG-Rhodamine, M-PEG-DSPE, DPPE-PEG, DPPE-PEG-Amine, DPPE-PEG-Acid, DPPE-PEG-COOH, DPPE-PEG-CH₂COOH, DPPE-PEG-NHS, DPPE-PEG-Azide, DPPE-PEG-DBCO, DPPE-PEG-Aldehyde, DPPE-PEG-Biotin, DPPE-PEG-Folate, DPPE-PEG-OH, DPPE-PEG-SH, DPPE-PEG-SPDP, DPPE-PEG-IA, DPPE-PEG-vinylsulfone, DPPE-PEG-Cy3, DPPE-PEG-Cy5, DPPE-PEG-FITC, DPPE-PEG-Rhodamine, M-PEG-DPPE; DLin-DMA, DLin-D-DMA, DLin-MC3-DMA, DLin-KC2-DMA, DODMA, DOTAP, SM-102, L319.

12. The immunogenic composition of claim 1, wherein the first nucleic acid comprises a single stranded DNA.

13. The immunogenic composition of claim 12, wherein the first nucleic acid comprises an unmethylated cytosine (C)-phosphate (p)-guanosine (G) (CpG) oligodeoxyribonucleotide.

14. The immunogenic composition of claim 13, wherein the CpG oligodeoxyribonucleotide is a toll-like receptor-9 (TLR-9) agonist.

15. The immunogenic composition of claim 12, wherein the first nucleic acid can bind to the DEC-205 receptor on dendritic cells (DCs).

16. The immunogenic composition of claim 1, wherein the first nucleic acid comprise sequence that has at least 80 percent identify to SEQ ID NO: 5, SEQ ID NO: 6, SEQ ID NO: 7, or SEQ ID NO: 8.

17. The immunogenic composition of claim 16, wherein the first nucleic acid comprises SEQ ID NO: 5, SEQ ID NO: 6, SEQ ID NO: 7, SEQ ID NO: 8, or a combination thereof.

18. The immunogenic composition of claim 1, wherein the peptide comprises a neoantigen or a tumor cell neoepitope present, or a fragment thereof.

19. The immunogenic composition of claim 18, wherein the tumor cell is a pancreatic tumor cell.

20-28. (canceled)

29. An vaccine composition comprising:
a) a lipid vesicle comprising one or more lipid components;
b) a first nucleic acid;
c) a peptide; and
d) one or more anchoring motifs containing one or more hydrophobic tails,
wherein the first nucleic acid forms a conjugate with the one or more anchoring motifs.

30-56. (canceled)

57. A method of preparing an immunogenic composition or vaccine composition of claim 1, the method comprising:
providing the lipid vesicle, the first nucleic acid, and the peptide;
optionally forming a conjugate by attaching the peptide to the second nucleic acid;
forming a complex of the first nucleic acid and the peptide or a hybridized complex of the first nucleic acid and the conjugate; and
forming a nanoparticle by combining the lipid vesicle and the complex or the hybridized complex.

58-59. (canceled)

60. A method of treating or preventing a cancer in a subject comprising administering an immunogenic composition or vaccine composition of claim 1.

61-65. (canceled)

66. A kit comprising an immunogenic composition or vaccine composition of claim 1.

67-68. (canceled)


SEQUENCE LISTING (8 sequences)

SEQ ID NO: 1 - protein, 20 aa, Mus sp.
GSSAEESHLSCLNWSTLVPL

SEQ ID NO: 2 - protein, 20 aa, Mus sp.
IKIVRLTTGSAYQFRVCAEN

SEQ ID NO: 3 - DNA, 20 bp, synthetic construct
  source 1..20: mol_type = other DNA
  modified_base 20: mod_base = OTHER; note = Thiol modified nucleotide
aacgtcaggaacgtcatgga

SEQ ID NO: 4 - DNA, 20 bp, synthetic construct
  source 1..20: mol_type = other DNA
  modified_base 1: mod_base = OTHER; note = Thiol modified nucleotide
aacgtcaggaacgtcatgga

SEQ ID NO: 5 - DNA, 28 bp, synthetic construct
  source 1..28: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
  modified_base 24: mod_base = OTHER; note = Nucleotide with phosphorothioate linkage
  modified_base 25: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Phosphorothioate amine modified nucleotide
tccatgacgttcctgacgtttttttttt

SEQ ID NO: 6 - DNA, 32 bp, synthetic construct
  source 1..32: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
  modified_base 24: mod_base = OTHER; note = Nucleotide with phosphorothioate linkage
  modified_base 25: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 29: mod_base = OTHER; note = Nucleotide with phosphorothioate linkage
  modified_base 30: mod_base = OTHER; note = Nucleotide with phosphorothioate linkage
  modified_base 31: mod_base = OTHER; note = Nucleotide with phosphorothioate linkage
  modified_base 32: mod_base = OTHER; note = Phosphorothioate amine modified nucleotide
tcgtcgttttgtcgttttgtcgtttttttttt

SEQ ID NO: 7 - DNA, 55 bp, synthetic construct
  source 1..55: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
  modified_base 25: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 30: mod_base = OTHER; note = Nucleotide with phosphorothioate linkage
  modified_base 31: mod_base = OTHER; note = Nucleotide with phosphorothioate linkage
  modified_base 33: mod_base = OTHER; note = Nucleotide with phosphorothioate linkage
  modified_base 34: mod_base = OTHER; note = Nucleotide with phosphorothioate linkage
  modified_base 35: mod_base = OTHER; note = Nucleotide with phosphorothioate linkage
  modified_base 37: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage
  modified_base 40: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage
  modified_base 48: mod_base = OTHER; note = Nucleotide with phosphorothioate linkage
  modified_base 49: mod_base = OTHER; note = Nucleotide with phosphorothioate linkage
  modified_base 50: mod_base = OTHER; note = Nucleotide with phosphorothioate linkage
  modified_base 51: mod_base = OTHER; note = Nucleotide with phosphorothioate linkage
  modified_base 52: mod_base = OTHER; note = Nucleotide with phosphorothioate linkage
  modified_base 53: mod_base = OTHER; note = Nucleotide with phosphorothioate linkage
  modified_base 54: mod_base = OTHER; note = Nucleotide with phosphorothioate linkage
  modified_base 55: mod_base = OTHER; note = Phosphorothioate amine modified nucleotide
tcgtcgttttgtcgttttgtcgtttcgtcgacgatcggcgcgcgccgtttttttt

SEQ ID NO: 8 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Aminated nucleotide
tttttttttttttttttttt